(19) United States
(12) Patent Application Publication
(10) Pub. No.: US 2007/0233089 A1
(43) Pub. Date: Oct. 4, 2007

(54) SYSTEMS AND METHODS FOR REDUCING ADJACENT LEVEL DISC DISEASE

(75) Inventors: Gene P. DiPoto, Upton, MA (US); Alan E. Shluzas, West Roxbury, MA (US)

Correspondence Address: KNOBBE MARTENS OLSON & BEAR LLP, 2040 MAIN STREET, FOURTEENTH FLOOR, IRVINE, CA, 92614, US

(73) Assignee: ENDIUS, INC., Plainville, MA (US)

(21) Appl. No.: 11/676,484

(22) Filed: Feb. 19, 2007

Related U.S. Application Data
(60) Provisional application No. 60/774,320, filed on Feb. 17, 2006.

(51) Int. Cl. A61F 2/30

(52) U.S. Cl. 606/61

(57) ABSTRACT

A spacer device is provided for use with a primary spinal fixation device to treat, reduce, or delay adjacent level degenerative disc disease. The spacer device comprises a compressible spacer, a transverse member, and a connecting member. The compressible spacer is sized to fit between the spinous processes of two adjacent vertebrae and is configured to reduce the range of motion of at least one vertebra. The transverse member is configured to extend from one side of the midline of the spine, extending through the interspinous process space. The transverse member is coupled with the spacer. The connecting member is attachable to the transverse member and to a primary spinal fixation device.

### CROSS-REFERENCE TO RELATED APPLICATIONS

This application claims the benefit of priority from U.S. Provisional No. 60/774,320, filed Feb. 17, 2006, which is incorporated by reference herein.

### BACKGROUND OF THE INVENTION

1. Field of the Invention

This application relates to apparatus and methods for providing support to one or more vertebrae that are adjacent to a surgical site, e.g., to reduce adjacent level disc disease.

2. Description of the Related Art

A common procedure for treating degenerative disc disease (DDD) is fusing or fixing together two or more vertebrae at the affected level or levels of the spine. However, many patients who have undergone a fusion or fixation procedure experience degeneration of the spinal segments (e.g., discs, vertebrae, and nerves) adjacent to the fusion or fixation site. This adjacent level degenerative disc disease can occur soon after surgery, e.g., within five years of the primary fusion or fixation procedure.

Adjacent level DDD is currently treated by performing a second fusion or fixation procedure at one or more levels adjacent to the primary fusion or fixation levels. The second procedure requires another operation on the patient and an extension of the fusion or fixation hardware to the affected level(s). In many cases, the second operation requires disassembly of some of the hardware from the site of the primary procedure. However, this hardware can be partially or completely encapsulated by bridging bone and/or scar tissue, which makes the second operation more difficult. Trauma caused by retraction of the musculature at the primary site can cause further damage to the tissue. The second operation causes significant trauma and discomfort to the patient.

### SUMMARY OF THE INVENTION

Accordingly, there is a need for apparatus, systems, and methods that can eliminate, slow, or stop the progress of adjacent level DDD to reduce the patient's chances of requiring a second fusion, fixation or other operative procedure of the body adjacent to, and including, the spine.

In one embodiment for treating the spine, a crosslink spacer device can be implanted to support and stabilize adjacent levels to the primary fixation or fusion site. In one embodiment, the crosslink spacer device comprises a spacer rod, a crosslink spacer, and a connecting member to attach the device to the primary fusion or fixation hardware. In some embodiments, the crosslink spacer is positioned between the spinous processes of the primary and adjacent levels to limit the compression of the vertebrae.

In one embodiment, a device for supporting a spinal segment of a patient is provided that includes one or more spacer rods, one or more spacers configured to be coupled with the one or more spacer rods and configured to support and stabilize adjacent vertebrae. The device also includes one or more connecting members configured to couple the one or more spacer rods to the spine of the patient. The spacer is capable of being positioned between adjacent spinous processes of the spine of the patient.

In one another embodiment, a device is provided for supporting spinal anatomy adjacent to a spinal segment for which normal range of motion is compromised. The device comprises a spacer configured to be positioned between a spinal segment, e.g., a portion of a vertebra such as a spinous process or lamina, of one of a plurality of affected vertebrae and a spinal segment of another vertebra adjacent to the affected vertebrae. The device can be configured to extend between the spinal segment of one of a plurality of affected vertebrae and the spinal segment of the adjacent vertebra.

In another embodiment a method is provided for reducing, delaying, or eliminating adjacent level DDD. The method involves accessing a region of the spine where normal range of motion is compromised, e.g., as in a fusion procedure. A spacer is positioned between a vertebral portion of one of the vertebrae for which the normal range of motion is compromised and a corresponding vertebral portion of an adjacent vertebrae, e.g., between adjacent spinous processes or lamina. The spacer can be coupled with a fixation assembly, e.g., by a rigid member such as a rod. The spacer can be movably coupled using a device such as a ball joint to permit some motion of the spacer relative to the fused spinal segment or between the spacer and a fixation or motion limiting device coupled with the affected spinal segment.

In another embodiment a spinal stabilization apparatus comprises a primary stabilization device and a device configured to intermittently interact with an adjacent spinal level. The primary stabilization device comprises a first screw configured to be inserted into a first vertebra and a second screw configured to be inserted into a second vertebra. The primary stabilization device also comprises a first elongate member extendable between the first and second screws. The first elongate member is configured to reduce at least some of the range of motion of the first and second vertebrae. The device is configured to intermittently interact with an adjacent spinal level comprises a spacer and a second elongate member. The spacer is configured to be inserted between a spinous process of the first vertebra and a second vertebra adjacent to the first vertebra. The second elongate member is configured to interconnect the spacer and the primary stabilization device.

In another embodiment an apparatus is provided for reducing adjacent level disc disease. The apparatus comprises a fixation device and a device configured to reduce adjacent level disc disease. The fixation device comprises a first screw configured to be inserted into a first vertebra and a second screw configured to be inserted into a second vertebra. The device configured to reduce adjacent level disc disease comprises a spacer and an elongate member. The spacer is configured to be inserted between a spinous process of the second vertebra and a third vertebra adjacent to the second vertebra. The elongate member is configured to interconnect the spacer and the fixation device.

In another embodiment a spacer device is provided for use with a primary spinal fixation device. The spacer device comprises a compressible spacer, a transverse member, and a connecting member. The compressible spacer is sized to fit between the spinous processes of two lumbar vertebrae and is configured to reduce the range of motion of at least one vertebra. The transverse member is configured to extend from one side of the midline of the spine, extending through the interspinous process space. The transverse member is coupled with the spacer. The connecting member is attachable to the transverse member and to a primary spinal fixation device.

In another embodiment, a method is provided for reducing or delaying degenerative disc disease. The method involves accessing a region of the spine where normal range of motion is compromised. A spacer is placed between a vertebral portion of one of the vertebrae for which the normal range of motion is compromised and a corresponding vertebral portion of an adjacent vertebrae. The spacer is coupled with a fixation assembly by a rod.

In another embodiment, a method is provided for treating a spine of a patient. The method involves inserting an access device through a minimally invasive incision in the skin of the patient. The access device is advanced until a distal portion thereof is located adjacent the spine. The access device is expanded from a first configuration to a second configuration. The second configuration has an enlarged cross-sectional area at the distal portion thereof such that the distal portion extends across at least two of three adjacent vertebrae. A first device is delivered through the access device to a location between a first pair of adjacent vertebrae. The first device is configured to preserve motion between the first pair of adjacent vertebrae. A second device is delivered through the access device to a location between a second pair of adjacent vertebrae. The second device is configured to preserve motion between the second pair of adjacent vertebrae.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention.

FIG. 1 illustrates an embodiment of an adjacent level device providing support for a spinal segment adjacent to a primary fusion or fixation site on a patient's spine.

FIGS. 1A-1H illustrate various embodiments of a spacer on various embodiments of a spacer rod.

FIG. 2 is a perspective view of an embodiment of a device for reducing adjacent level disc disease that can include a spacer apparatus and a ball joint connecting member.

FIG. 3 is a perspective view of another embodiment of a device for reducing adjacent level disc disease that can include a spacer apparatus and a connecting member.

FIG. 4A is a perspective view of an embodiment of a connecting member or device.

FIG. 4B is an end view taken of the connecting member or device illustrated in FIG. 4A.

FIG. 5A is a perspective view of one embodiment of a device for reducing adjacent level disc disease, including a connecting member similar to that shown in FIG. 4.

FIG. 5B is a perspective view of another embodiment of a device for reducing adjacent level disc disease, including a connecting member or device that includes a ball joint.

FIG. 6 is a top view of one embodiment of a device for reducing adjacent level disc disease with a lateral connecting member.

FIG. 6A is a cross-sectional view of one open embodiment of a lateral connecting member as shown in FIG. 6.

FIG. 6B is a cross-sectional view of one closed embodiment of a lateral connecting member as shown in FIG. 6.

FIG. 7A is a perspective view of a portion of an embodiment of a connecting member with a bone screw.

FIG. 7B is an exploded perspective view of the embodiment of a connecting member with a bone screw illustrated in FIG. 7A.

FIG. 8 is a perspective view of one embodiment of an access device.

FIG. 9 is a schematic view of one surface of a vertebra and various approaches for spinal access of an access device configured to provide access, e.g. an access path, to the vertebra.

FIG. 10 is a schematic view of one surface of a vertebra and one embodiment of an access device configured to provide access to the vertebra or the space around the vertebra.

FIG. 11 is a partial sectional view of a stage of one embodiment of a method for treating the spine of a patient;

FIG. 12 is a partial sectional view of another stage of one embodiment of a method for treating the spine of a patient;

FIG. 13 is a partial sectional view of a stage of one embodiment of a method for treating the spine of a patient with an adjacent level device comprising a connecting member;

FIG. 14 is a partial sectional view of a stage of one embodiment of a method for treating the spine of a patient with an adjacent level device comprising a connecting member with a bone screw as illustrated in FIG. 7A-7B;

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF PREFERRED EMBODIMENTS

As should be understood in view of the following detailed description, this application is primarily directed to apparatuses and methods for treating the spine of a patient. The apparatuses described below can be configured to provide a variety of treatments to reduce or delay degenerative disc disease (DDD) in or near the spine of a patient. In particular, various embodiments described herein below can include devices for fusion, fixation, limiting motion, or providing dynamic support of one or more levels of the spine and structures adjacent to or near the spine. Various methods are disclosed for working with these apparatuses. The apparatuses and methods described enable a surgeon to perform a wide variety of methods of treatment for reducing or delaying adjacent level DDD of a patient as described herein. Such apparatuses can be deployed through an access device that at least partially defines an access path through otherwise naturally continuous tissue from outside the patient to the spine. Such an access device preferably would provide minimally invasive access, but the apparatuses and methods described herein are applicable to open surgery as well.

A. Apparatuses for Treating and Reducing Adjacent Level Degenerative Disc Disease

The degeneration of spinal segments, such as vertebral levels, vertebrae, discs, and nerves, adjacent to a spinal segment where a primary fusion, stabilization or fixation procedure has been performed can be caused by one or more of a concentration of force and certain types of movement of the adjacent level(s) as a result of the restriction of movement of the fused or fixated level or levels. This application discusses devices that can reduce at least one of the concentration of force on and particular types of movement of adjacent level spinal segments. Adjacent and primary sites can include joints between any of the cervical, thoracic, lumbar, and sacral vertebrae, as well as the joint between the skull and the first cervical vertebra (skull-C1). Such devices can slow down or substantially prevent the degeneration known as adjacent level DDD. For example, an adjacent level device can be configured to restrict the compression, flexion, or torsion of the spinal segments, e.g., vertebral levels adjacent to the primary treatment (e.g., fixation) site and to provide additional dynamic support to the adjacent levels. Implanting the adjacent level device during the initial fusion or fixation procedure can beneficially delay or substantially prevent the onset of adjacent level DDD. Implanting the adjacent level device during the initial fusion, fixation, or other adjacent procedure can advantageously eliminate or reduce the need for a second operation to treat adjacent spinal segments, e.g., to fuse or fix adjacent levels that have collapsed or degenerated. Further, embodiments of the device that are sized and shaped so as to be capable of being implanted during a minimally invasive surgical procedure will beneficially reduce operative and post-operative trauma to the patient.

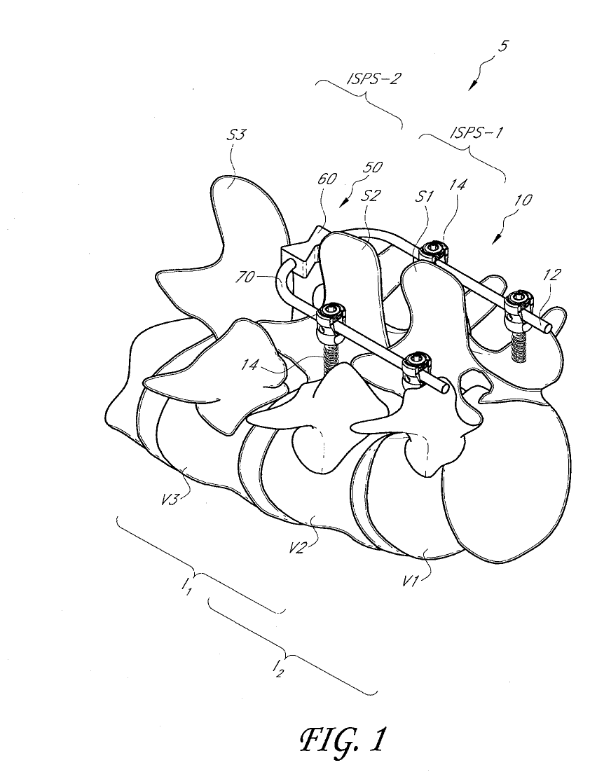

FIG. 1 illustrates a perspective view of a spinal segment, e.g., a portion of the spine of a patient, showing vertebrae V1, V2, and V3 and spinous processes S1, S2, and S3. A first intervertebral region I1 is located at least partially between V1 and V2. An interspinous process space (ISPS) is at least partially located between adjacent interspinous processes. A first interspinous process space ISPS-1 is located within intervertebral region I1. An intervertebral region I2 is located at least partially between V2 and V3. A second interspinous process space ISPS-2 is located within intervertebral region I2. In the illustrated example, the spinal segment comprising vertebrae V1 and V2 is damaged and requires fusion, stabilization, fixation or some other treatment including motion preservation devices and treatments.

Accordingly, one embodiment of an adjacent level device 5 which comprises a spacer device 50 is implantable in the patient. The adjacent level device 5 can be configured to connect with or be coupled with a stabilization device 10. In some embodiments, the stabilization device 10, which can include pedicle screws and fixation rod(s), as discussed below, can form a part of a device counteracting adjacent level DDD.

In one embodiment a stabilization device 10 is adapted to be secured to vertebrae V1 and V2. In various embodiments, the stabilization device 10 is a fixation device, a fusion device, or a dynamic stabilization device. As shown in FIG. 1, the stabilization device 10 is a fixation device, which comprises an elongate element 12 and a fastener assembly 14. In various embodiments, the elongate element 12 is a fixation rod, a fusion rod, a stabilization rod, a fixation plate, or an elongate member. As discussed below, the stabilization device 10 need not include all of these components. For example, the elongate element 12 need not be included in transfacet or translaminal fixation. In various embodiments, the fastener assembly 14 includes one or more screws that can be attached to the vertebral body, pedicle, or lamina of vertebrae V1 and V2 of the patient. FIG. 1 illustrates a procedure in which the screws of a fastener assembly 14 are inserted into pedicles of the vertebrae V1 and V2 to provide a stable construct. As illustrated, an embodiment of the fastener assembly 14 shows a portion of threads from a screw for the purposes of illustration. When fully installed, the threads are advanced more completely into bone. In other embodiments the stabilization device 10 can be attached to other portions of a spine.

The elongate element 12 can take any suitable form, for example, being stiff enough to assure that there is no motion between the vertebrae V1, V2 or to be flexible to permit some motion, e.g., in providing dynamic stabilization with at least a fraction of the normal range of motion. This fixation procedure can be accompanied by a procedure in which a fusion device is inserted between the vertebrae V1, V2. In other embodiments, different or multiple stabilization devices 10 can be used, and the stabilization device 10 can comprise additional or different components, e.g., more screws and longer rods for multi-level fixation or other hardware discussed below.

The spacer device 5 can be installed in patients where degeneration of adjacent spinal segments, (e.g., a vertebral level including vertebra V3) could occur. The spacer device 50 can be configured to reduce motion or force, particularly the concentration of force due the presence of a fixation or other stabilization device, on the adjacent spinal segment.

In one embodiment, the spacer device 50 comprises a spacer 60 and a spacer rod 70 configured to position the spacer 60 between spinous processes S2 and S3 of the vertebrae V2 and V3, respectively. In one embodiment, the spacer device 50 is a crosslink spacer assembly or a crosslink spacer device. The spacer device 50 can be moveably or fixedly coupled to one or more stabilization devices 10 in one, two, or any number of places. For example, the spacer device 50 can be moveably or fixedly attached to a stabilization device 10 on one side of a spinous process with a single spacer rod 70. In some embodiments the spacer device 50 is configured to be attached to the spine with its own fasteners, such as a screw or a connecting member with a screw and housing. Such an arrangement can still include an elongate member similar to the spacer rod 70 that interconnects the spacer 60 with the screw or other implant to be coupled with the spine.

FIG. 1 shows that the adjacent level device 5 can comprise a spacer device 50 and a stabilization device 10. In one variation, the elongate element 12 comprises a stabilization rod and the fastener assembly 14 comprises a screw. In one embodiment, the spacer rod 70 and the stabilization rod are substantially continuous portions of a rod that is bent into a “U” shape. In various embodiments, the spacer rod 70 is an elongate member or a transverse member. In some embodiments the spacer rod 70 can be shaped like a “U”, half of a “U”, or a curve or arc. The spacer rod 70 can be configured to be assembled with the stabilization rod, as discussed below. The spacer rod 70 and/or the elongate member 12 can be pre-bent or bent to fit during the implantation procedure based on the patient's anatomy. The spacer rod 70 can comprise the same material as the elongate member 12. In other embodiments, however, the spacer rod 70 comprises a different material that can be selected for the elongate member 12 to provide differing levels of dynamic stabilization or motion reduction for the adjacent levels. In some embodiments, the spacer rod 70 comprises a biocompatible metal such as, for example, titanium. Other materials are possible such as, for example, Nitinol or a polymer, e.g., polyetheretherketone (PEEK) or polyethyleneterephthalate (PET). Although referred to herein as a rod, in various embodiments the spacer rod 70 can be any form of appropriate elongate element or elongate member, such as a tether, rope, chain, ribbon, or film which can be flexible. In some embodiments the spacer rod 70 can be threadable through a ligament or other tissue, such as by twisting or applying pressure on a sharp point on the spacer rod 70 in order to advance the spacer rod 70 through the tissue.

In some embodiments, the spacer rod 70 can be any form of elongate element that holds a spacer 60 in a desired orientation within an intervertebral region, such as in I2 or such as between two spinous processes, e.g., between a spinous process associated with a vertebra that has been fixed or fused and a spinous process above or below the fixed or fused vertebra. In some embodiments the spacer rod 70 is configured, e.g., is sized or rigid enough, to hold the spacer 60 in an interspinous process space, such as ISPS-2. The spacer 60 can be positioned between adjacent spinous processes and, at various times depending on the flexion or position of the spine of the patient, the spacer 60 can touch one, both, or neither of the adjacent spinous processes.

The spacer 60 can be moveably or fixedly coupled to the spacer rod 70. In certain embodiments, the spacer rod 70 is welded, bonded or adhered to the spacer 60. In other embodiments, the spacer rod 70 can be secured to the spacer 60 by one or more connectors such as, for example, screws or rivets. In other embodiments, the spacer rod 70 is inserted into or through a passageway that extends within or entirely through the spacer 60. In other embodiments the spacer rod 70 has a texture or surface treatment that increases the friction between the rod 70 and the spacer 60, e.g., to hold the spacer 60 in a particular location along the rod 70. In other embodiments, the stabilization elongate element 12 can be welded, bonded or adhered to the spacer 60. In other embodiments where the stabilization elongate element 12 is contiguous with or forms a part of the spacer rod 70, the stabilization elongate element 12 can be secured to the spacer 60 by one or more connectors such as, for example, screws or rivets. In other embodiments, the stabilization elongate element 12 is inserted through a passageway within the spacer 60.

As shown in FIG. 1, the spacer 60 is disposed between the spinous processes S2 and S3 of the adjacent vertebrae V2 and V3. In other embodiments, the spacer 60 can be disposed in other locations such as, for example, between the lamina or other bony segments that are strong enough to transmit forces related to spinal segment motion without being damaged. The spacer 60 can be configured to inhibit the compression of the spine by reducing the range of motion over which, in one embodiment, the spinous processes S2 and S3 can approach each other. In the embodiment shown in FIG. 1, the spacer 60 provides minimal restriction on the flexion of the spinous processes S2 and S3. However, other embodiments could be used in combination with the embodiments described above to reduce flexion, e.g., by flexibly or rigidly tethering, adhering to, gripping or hooking at least a portion of the spinous process S3.

The configuration, e.g., the size, shape, and material properties of the spacer 60, are selected to provide a suitable amount of support for the adjacent spinal segment(s) to reduce the concentration of force on these segments due to the primary stabilization, fusion, or fixation. In some embodiments, the spacer 60 can be hollow. In some embodiments the spacer 60 can be made of a combination of materials. In some embodiments, the spacer 60 can be a spring, a resilient member, or a compressible member, e.g. one that will compress under normal loading conditions of the spine. In some embodiments, the spacer 60 can be a resilient member that can be compressed up to about 25% of its unloaded shape or size (e.g., transverse size) when subject to normal loading, such as in walking twisting, jumping, running, or other typical activities. In some cases, the spacer 60 is a resilient member that can be compressed up to about 50% under such normal conditions. In some cases, the spacer 60 is a resilient member that can be compressed by 50% or more than 50% under such normal conditions. In other embodiments the spacer can not significantly deflect or compress under normal spinal loading. In FIG. 1, the spacer 60 has a general “bow-tie” shape. The spacer 60 can be configured with a narrowing near a central portion thereof and a widening on at least one peripheral side. The narrowing and widening are suitable ways to orient and help maintain the position of the spacer 60. For example, the inferior narrowing near a central portion of the spacer 60 can house the superior surface of an inferior spinous process by abutting each of the lateral sides of the inferior spinous process with the widened portion of the spacer 60. Likewise, a superior narrowing near a central portion of the spacer 60 can house the inferior surface of a superior spinous process by abutting each of the lateral sides of the superior spinous process with the widened portion of the spacer 60. This type of configuration is advantageous in that the axial motion of the spinous processes, such as through flexion of the spine from bending over, is relatively unhindered compared to the limitation to rotation of the spine by the lateral sides of the widened portions of the spacer 60. In other embodiments, the spacer 60 can only have a narrowing and widening on one of a superior or inferior surface of the spacer 60. In some embodiments the spacer 60 is adapted to fit securely between adjacent spinous processes S2 and S3. The spacer 60 may also be contoured or shaped to fit or mate closely with the anatomy between the spinous processes.

FIGS. 1A-1H show other embodiments in which the spacer 60 and the spacer rod 70 have different configurations. In some embodiments the spacer has a channel (not shown) through which the spacer rod extends. Many sizes, shapes, materials and combinations are possible. For example, FIG. 1A shows a spacer 60A that can have a shape similar to the bow-tie as described in FIG. 1 above. The configuration is similar to that of FIG. 1 except the superior surface of the spacer 60 is flatter than the narrowed surface of the inferior surface. The flatter surface allows a greater range of rotational motion of the superior spinous process with respect to the adjacent level device than is constricted by the narrower center and steeper widened portion sides. In various embodiments, the spacer 60 and 60A-60H can be symmetric or it may be non-symmetric in order to limit motion or increase shock absorption in a particular direction or orientation. For example, the flatter surface of the spacer 60A can be on the bottom (or inferior) surface instead of the top (or superior) surface. Likewise, the left or the right side may be flatter than the opposite side to allow more of a range of motion in one degree of rotation as compared to another. As illustrated in FIG. 1A, the spacer rod 70A can terminate in a sharp tip such as a blade or cone. This configuration is advantageous in that it may be used to pierce though the intraspinous ligament such as in a minimally invasive surgical (MIS) approach. Likewise the spacer 60A itself may terminate on one side in a cone or other tissue piercing shape in a manner similar to spacer 60H, described below.

FIG. 1B shows a spacer 60B that has the shape of a sphere. This configuration is advantageous in that the spacer 60B may provide support against axial compression of the spinous processes due to flexion of the spine (such as in bending backward) while leaving rotation of the spine relatively unhindered. In one embodiment a spacer rod 70B can have a stopping feature configured to hold a spacer (any spacer including 60B) from one side. In one embodiment the stopping feature is a stop 71 comprised of a diameter or dimensional feature which is greater than the size of the channel through the spacer which impedes the spacer from advancing in the direction of the stop 71. One advantage of this configuration is additional certainty in the placement of the spacer 60B with respect to the spacer rod 70B between spinous processes. As illustrated, the stop 71 terminates the spacer rod 70B such that the spacer rod 70B is attached to a connecting member (not illustrated) or fastening assembly (not illustrated) on one side of the spinous process. In other embodiments, stopping features, such as a stop 71, can be used where the spacer rod 70B continues past the stop 71 and can be connected to a connecting member (not illustrated) or fastening assembly (not illustrated) on a second side of the spinous process.

FIG. 1C shows a spacer 60C that can have the shape of an oval. This configuration is advantageous in that it provides a cushioning along a wider range of rotation than the spacer 60B of FIG. 1B, providing more of a reduction of concentrations of force along the spinous processes and the spine in general through a wider range of rotation of the spine. Variation in the combination of axial and rotational motion limitation of the spine along with variance in the level of cushioning or shock absorption desired through a range of motion can result in additional shapes of a spacer, including but not limited to an ellipsoid, an egg-shape, or a toroid. In one embodiment a spacer rod 70C can be a rope, thread, tether, or ribbon with or without a knot 72 adjacent to the spacer 60C. One advantage of this configuration is a higher degree of the potential range of motion in the spine that is allowed by flexible spacer rod 70C. The spacer rod 70C may be made of compliant materials such as plastic or metal wires. A flexible spacer rod 70C can also be easier to install or manipulate within the patient during implantation or removal of the device. A flexible spacer rod 70C can also have a lower profile and displace less surrounding tissue than a larger less-flexible rod or other extrusion.

FIG. 1D shows a spacer 60D that can have the shape of a conjoined orbs, and has many of the advantages of the bow-tie configuration of spacer 60 and spacer 60A described above. However, with more rounded edges, spacer 60D can provide for smoother rotation of the spine and spinous processes over the spacer 60D. In one embodiment a spacer rod 70D can be a chain or linkage which has many of the advantages of the flexible spacer rods 70C described above, but can be a more robust and less prone to wear if made of a metal such as Nitinol instead of a fiber or plastic in certain embodiments of rope or ribbon. The end of the chain or linkage can have a portion configured to connect to a connector, connecting member, or other device.

FIG. 1E shows a spacer 60E that can have the shape of a block with inset levels and has many of the advantages of the bow-tie configuration of spacer 60, spacer 60A, and spacer 60D described above but allows ranges of rotation with constant resistance as compared to the gradient of resistance that is provided by a spacer with sloped or rounded sides. The flat superior and inferior surfaces also provide a larger area over which loads can be transmitted between the spinous process and the spacer 60E, thereby reducing pressure on the surface of the spinous processes. In one embodiment a spacer rod 70E can have any variety of snap fit or bayonet feature configured to connect with a connecting member, connector block, fixture or screw. One advantage of this configuration is the increased speed and ease of assembly of the adjacent level device within the patient.

FIG. 1F shows a spacer 60F that can have the shape of a series of cylinders with varying radii. In addition to the advantages of similarly shaped and described spacers above, the rounded feature of the various radii extending from the axis of the spacer 60F allow for a greater range of rotation circumferentially to the axis of the spacer 60F that would otherwise be impeded by the flatter superior and inferior surfaces of a spacer similar to spacer 60E. Spacer rod 70F can comprise a spring, elastic member, or a flexible member which can in certain embodiments be connected between rods. One advantage of this configuration is to allow for alignment and/or mobility of the segment.

FIG. 1G shows a spacer 60G that can have the shape of a profile that is substantially the same as the profile of the spacer rod 70G. One advantage of maintaining a lower profile spacer as with spacer 70F is the ability to fit the adjacent level device into smaller regions of the spine, such as the cervical spine, or to treat adjacent level DDD where a smaller limit to motion or a slight amount of force absorption is needed relative to conditions with the larger diameter or dimensioned spacers. Spacer rod 70G can be a rod of circular, oval, square, rectangular, or other cross-sectional profile appropriate for the spinal geometry.

FIG. 1H shows a spacer 60H that in one embodiment can comprise at least two conical surfaces. Two symmetric or non-symmetrical conical surfaces can be oriented to point toward each other in order to create a narrowing toward the middle of the spacer. One of the advantages of this configuration is the higher or wider sides help restrict rotational motion of the spine while allowing relatively unrestricted motion to flexion of the spine as described in some of the “bow-tie” embodiments described herein. Another advantage of the rounded surface feature of the gradually ascending radii extending from the axis of the spacer 60H is that the spacer 60H allows for a greater range of rotation circumferentially to the axis of the spacer 60H that would otherwise be impeded by the flatter superior and inferior surfaces of a spacer similar to spacer 60E. As illustrated in FIG. 1H, an additional embodiment feature of the spacer 60H can include a sharp tip such as a cone on one side of the spacer. This configuration is advantageous in that it may be used to pierce though the intraspinous ligament such as in a minimally invasive surgical (MIS) approach. Any of the spacers described herein can have this sharpened feature. As illustrated, embodiments of the spacer rod 70H which are configured to work with a sharpened spacer can be attached to extend from only one side of the spacer.

In some embodiments the spacer rod 70 and 70A-70H can be elastic, axially elongatable, or compressible. In some embodiments the spacer rod 70 and 70A-70H comprises biocompatible flexible fibers such as, for example, natural or artificial ligaments. In some embodiments, the spacer 60 and 60A-60H can be a spring configured to interact with one or two spinous processes, where the spring can be shaped in a manner similar to spacer 60A in FIG. 1A or spacer 60E in FIG. 1E. In some embodiments, the spacer 60 and 60A-60H can be a spring that has projections configured to contact or lock on to one or two spinous processes. Any of the embodiments and feature or sub-features of the embodiments of the spacer 60 and 60A-60H can be used in any combination with any of the embodiments and feature or sub-features of the embodiments of the spacer rod 70 and 70A-70H disclosed herein.

The spacer 60 can comprise either a rigid or an elastic material depending on the amount of movement reduction desired for the adjacent levels. In certain embodiments the spacer 60 can comprise a biocompatible metal such as, for example, titanium, or the spacer 60 can comprise a biocompatible polymer (such as PEEK) or an elastomeric material. In some embodiments, the spacer 60 is configured so that minimal bone growth will occur between the spacer and adjacent bony segments, such as the spinous processes S2, S3.

In some embodiments, the spacer device 50 is sized and shaped to be implanted during a minimally invasive surgical procedure. It is preferable, although not necessary, for the spacer device 50 to be implanted during a fusion or fixation procedure that is performed at the same time (sometimes referred to herein as the “primary” fixation or fusion) so as to minimize trauma and to eliminate or slow the onset of adjacent level DDD. However, the spacer device 50 can also be implanted during a later surgical procedure. Although FIG. 1 shows one spacer device 50, more than one spacer device 50 can be used in patients. For example, one or more spacer devices 50 can be installed at vertebral levels above and/or below the primary stabilization, fusion, or fixation level. Further, one or more spacer devices 50 can be installed to support or stabilize vertebral levels that not immediately adjacent to the primary stabilization level, but that are located farther away from the primary site. In addition, each of the implanted spacer devices 50 can be selected to have suitable size, shape, and stabilization characteristics, and each need not be substantially the same. In some cases, the spacer device 50 can form a portion of a kit that is configured to enable a surgeon to treat all regions of the spine. For example, the spacer device 50 can be specifically configured for lumbar anatomy and can be included with similar devices that are specifically configured for the cervical, thoracic, or sacral regions.

The embodiment illustrated in FIG. 1 shows the spacer 60 inserted between the spinous processes S2 and S3 of an adjacent spinal segment (e.g., vertebral level). In other embodiments, the spacer 60 can be located and positioned differently. For example, in one embodiment a small or narrow spacer can be inserted between the lamina or the facets of the adjacent level. In one embodiment, a spacer sized and configured to be inserted in between the lamina or facets of adjacent spinal levels can be attached to a spacer rod in comprising a adjacent level device. In another embodiment, a spacer sized and configured to be inserted in between the lamina or facets of adjacent spinal levels can be attached in addition to an interspinous spacer or an interspinous process spacer. In one embodiment a lamina spacer or a facet spacer is attachable to a interspinous spacer rod, and in another embodiment a lamina spacer or a facet spacer is attachable to a connecting member or a fastening assembly. FIG. 1 illustrates an embodiment of the spacer device 50 that is connected to the stabilization device 10. In other embodiments, the spacer device 50 can be located, positioned, and attached differently. For example, the spacer device 50 can be tethered, secured, or fixated to a variety of locations at the surgical site including other suitable boney landmarks of the posterior vertebrae. Many variations are possible.

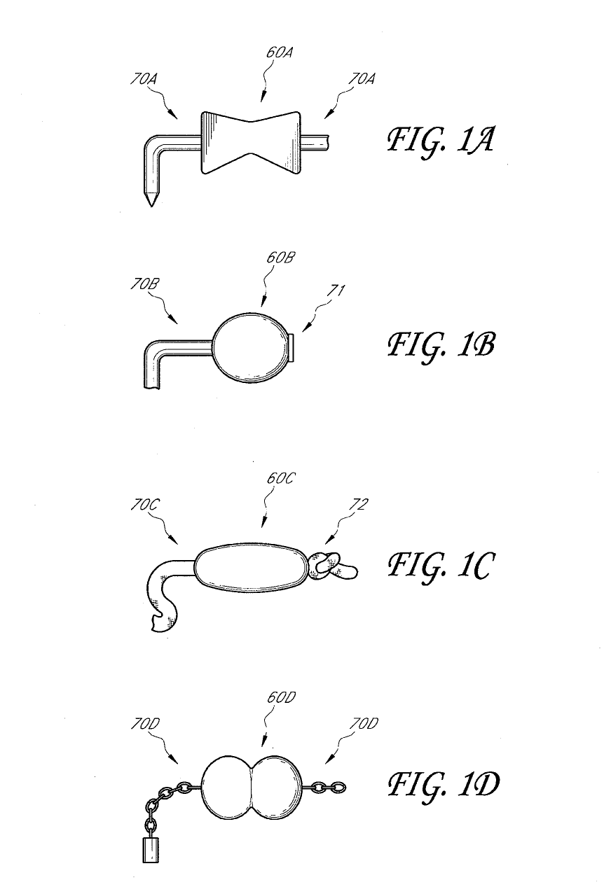

FIG. 2 illustrates another embodiment of a spacer device 52 that can include a spacer 60, a spacer rod 70 and a connecting member 30. In this embodiment, the connecting member 30 comprises a ball 32 and socket 31 joint. As shown in FIG. 2, the spacer rod 70 has a first end portion 71 and a spacer portion 72 near the spacer 60. The elongate element 12 of a stabilization device (the rest of the stabilization device is not illustrated here) comprises a first end portion 11 and a second end portion 13. The first end portion 71 of the spacer rod 70 comprises a socket 31 and the first end portion 11 of the elongate element 12 of a stabilization device comprises a ball 32. The ball 32 is adapted to engage the socket 31. In another arrangement, the first end portion 71 of the spacer rod 70 comprises the ball and the first end portion 11 of the elongate element 12 comprises the socket.

The connecting member 30 can be configured to provide a desired range of motion for the spacer device 52. For example, in one embodiment the ball 32 can be adapted to fit snugly within the socket 31 such that frictional engagement between the ball 32 and the socket 31 provides a suitable range of motion. For example, such friction can limit the range of motion of the ball 32 and/or the socket 31 or can absorb some of the energy that would otherwise be transferred to the spine or the spacer 60 coupled therewith. In other embodiments, the ball and socket joint can be clamped or otherwise configured to limit or restrict the range of motion.

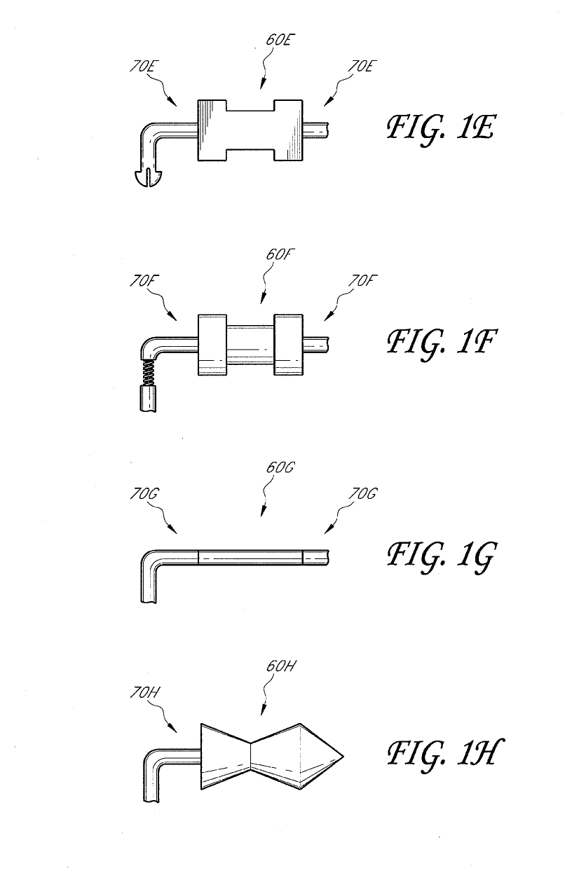

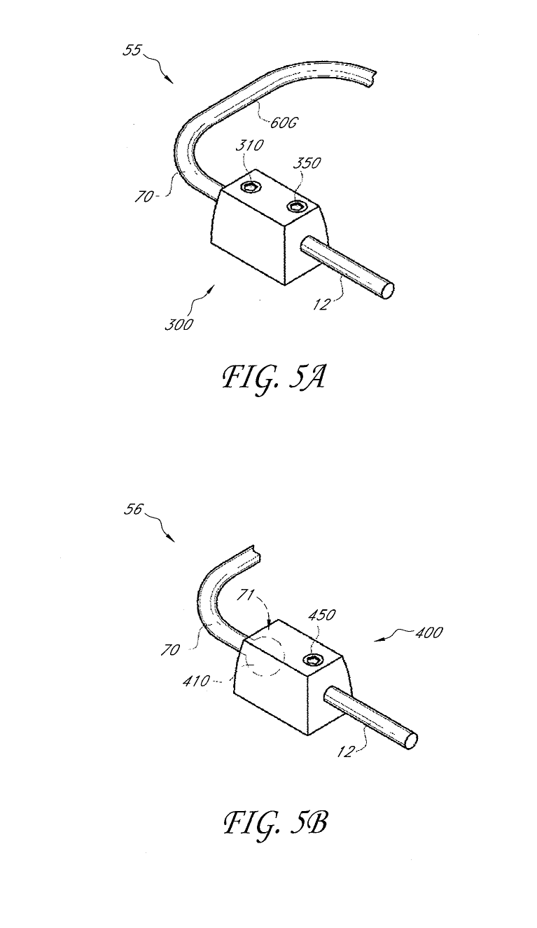

FIG. 3 illustrates another embodiment of a spacer device 53 comprising the spacer 60 and spacer rod 70 as described above. The spacer device 53 is configured to be attached to the elongate element 12 of the primary stabilization level by one or more connecting members 100. The connecting member 100 is configured to engage the spacer rod 70 and elongate element 12. The spacer rod 70 has a first end portion 71 which can be connected to or through a connecting member 100. The elongate element 12 of a stabilization device (the rest of the stabilization device is not illustrated here) comprises a first end portion 11 and a second end portion (not illustrated here). As shown in FIG. 3, the connecting member 100 comprises one or more passageways 120, 121 adapted to hold the spacer rod 70 and/or an elongate element 12. The passageway 120 is adapted to receive or to engage or to house either the spacer rod 70 and/or an elongate element 12. The passageway 121 is adapted to receive or to engage or to house either the spacer rod 70 and/or an elongate element 12. The passageways 120, 121 can be parallel, coaxially oriented, non-parallel, or can be arranged in various other orientations to accommodate the spinal geometry of the patient. In some embodiments, the connecting member 100 can comprise a single passageway for both the spacer rod 70 and the elongate element 12. (For example, one embodiment is shown in FIG. 5). This single passageway can be straight, coaxial, curved, and/or offset. The single passageway can be configured to accommodate different diameters or cross-sections to accommodate the profile of various sizes or shapes of spacer rods 70 and/or elongate elements 12. In other embodiments, a passageway (not illustrated) does not extend all the way through two surfaces of the connecting member, but instead terminates within the connecting member.

In one embodiment, the spacer rod 70 and/or the elongate element 12 can be secured by a clamping screw 110 that is configured to clamp directly on the spacer rod 70 and/or the elongate element 12 in a passageway 120 or 121, or in a separate, open- or close-ended channel (not illustrated). Other embodiments can utilize additional screws or clamps to position and secure the spacer rod 70 and/or the elongate element 12.

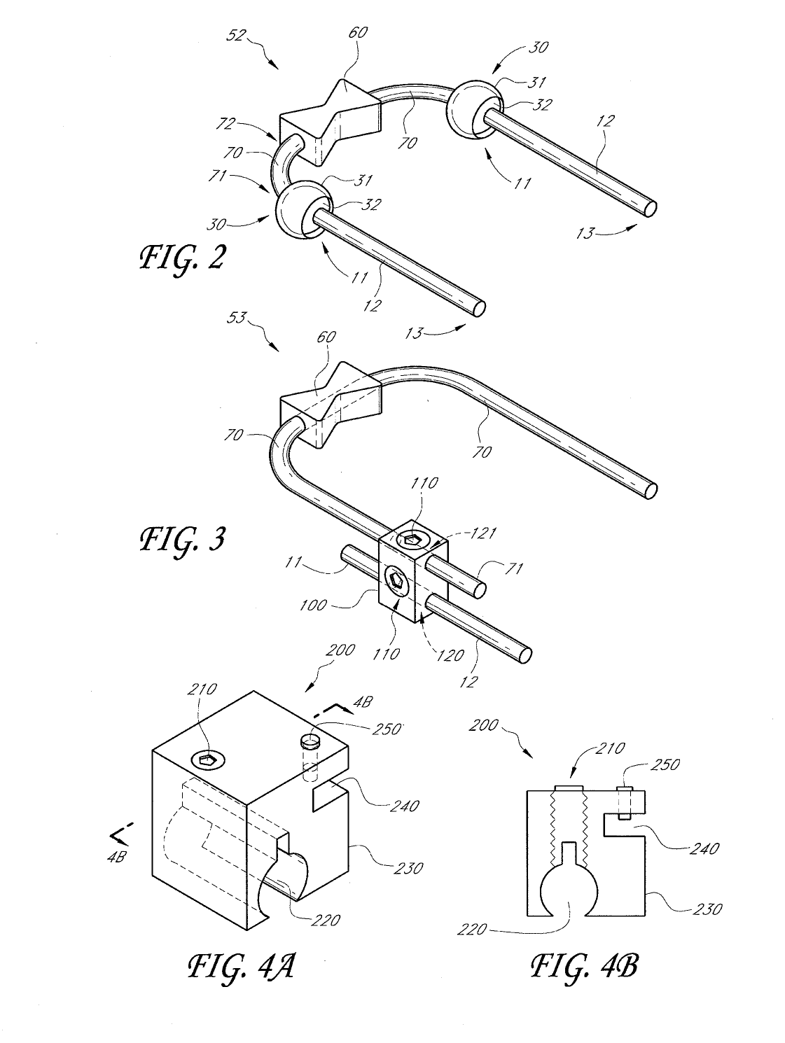

FIGS. 4A and 4B illustrate another embodiment of a connecting member 200. In this embodiment, the connecting member 200 comprises one or more passageways 220 adapted to receive or to engage an elongate element 12 (not shown). A wing portion 230 of the connecting member 200 is adapted to be sufficiently flexible such that an elongate element 12 can be snap-fit into the passageway 220. In some embodiments, the connecting member 200 is further configured to clamp the elongate element 12 into the passageway 220. For example, as shown in FIGS. 4A and 4B, a channel or groove 240 can be disposed above the wing portion 230. A set screw 250 is configured to engage a surface in the channel 240. The set screw 250 is rotated so that an end of the set screw 250 engages the surface of the channel 240 to urge the wing portion 230 to contact an elongate element 12 disposed within the passageway 220. By suitably tightening the set screw 250, the elongate element 12 can be secured or locked into a suitable position.

In certain embodiments, the spacer rod 70 (not illustrated) is secured to the connecting member 200 by a flexible wing portion, passageway, groove, and set screw that are substantially similar to those shown in FIGS. 4A and 4B and described above. In other embodiments, the spacer rod 70 and/or the elongate element 12 can be secured by a clamping screw 210 that is configured to clamp directly on the spacer rod 70 and/or the elongate element 12 in the passageway 220, or in a separate, open- or close-ended channel (not illustrated). Other embodiments can utilize additional screws or clamps to position and secure the spacer rod 70 and/or the elongate element 12. In one embodiment, both the spacer rod 70 and the elongate element 12 are clamped within the passageway 220.

FIG. 5A illustrates an embodiment of a spacer device 55 attached to an elongate element 12 by a connecting member 300. In FIG. 5A, the connecting member 300 is similar to the connecting members hereinbefore described. The connecting member 300 comprises a first clamping screw 310 that is used to secure a spacer rod 70 of a spacer device 55 and a second clamping screw 310 is used to secure an elongate element 12. In other embodiments, the connecting member 300 can comprise more than one clamping screw 310 to attach to at least one elongate element 12 and at least one spacer rod 70. In another embodiment (not illustrated), the connecting member 300 comprises a clamping screw 310 and a set screw 350 with a flexible wing portion, passageway, and groove that are substantially similar to those shown in FIGS. 4A and 4B and described above.

The spacer device 55 comprises a spacer 60G (see FIG. 1G) that is cylindrically shaped. The spacer 60G can comprise the same material as the spacer rod 70, or it can comprise different material. For example, the spacer rod 70 can comprise a metal, such as titanium, and the spacer 60G can comprise a more resilient material, such as an elastomer. In one embodiment the spacer 60G is highly compliant and acts as a shock absorber. In certain embodiments, the spacer 60G can have a diameter that is substantially similar to a diameter of the spacer rod 70. For example, in connection with cervical vertebrae, the separation between adjacent vertebrae is smaller than in the lumbar region. Accordingly, a smaller spacer 60G can be adequate to still reduce motion of forces somewhat. In other embodiments, the spacer has a different diameter than the spacer rod 70.

FIG. 5B illustrates another embodiment of a spacer device 56 attached to an elongate element 12 by a connecting member 400. In one embodiment, the elongate element 12 is secured to the connecting member 400 by a flexible wing (not visible) that is substantially similar to the flexible wing 230 illustrated in FIGS. 4A and 4B. The spacer rod 70 is attached to the connecting member 400 by a ball and socket joint 410. In this embodiment, a first end portion 71 of the spacer rod 70 comprises the ball, which is adapted to fit into the socket disposed in the connecting member 400. In various embodiments, the ball and socket joint 410 can be configured for any suitable range of motion, for example, permitting a wide range of smooth motion, a limited range of motion due to friction in the joint, or very little motion. In one embodiment the ball and socket joint 410 can be unclamped and utilize frictional engagement to provide sufficient stabilization and support for the adjacent vertebral levels or spinal segments. The joint 410 can be clamped by, for example, one or more screws.

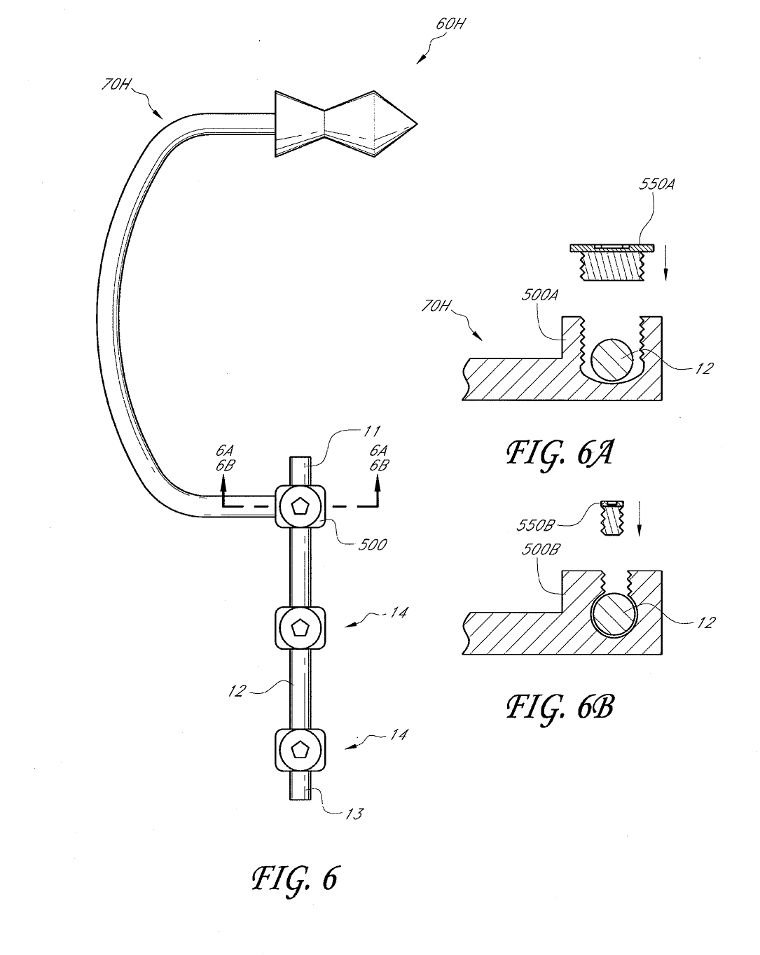

FIGS. 6, 6A and 6B illustrate another embodiment of an adjacent level device comprising any stabilization device and any spacer device embodiments described herein with a lateral connecting member 500. As illustrated, a stabilization device comprises an elongate element 12 with a first end portion 11 and a second end portion 13 and two fastening assemblies 14. A spacer device comprises a spacer 60H and a spacer rod 70H. Any embodiment can be used with the lateral connecting member 500. The lateral connecting member 500 can be attached to the first end portion 11 of the elongate element 12 of the stabilization device with an open configuration (FIG. 6A) or a closed configuration (FIG. 6B). In one embodiment the lateral connecting member 500 is an open lateral connecting member 500A which comprises an open channel in which an elongate member 12 can be secured by a clamping screw 550A. In another embodiment the lateral connecting member 500 is a closed lateral connecting member 500B which comprises a closed channel in which an elongate member 12 can be slidably inserted and secured by a clamping screw 550B.

Other embodiments of the connecting member can include a snap fit configured to work with a spacer rod 70E as shown in FIG. 1E. Certain embodiments of any of the spacer rods disclosed can have divots, interlocks, or features for engaging with a screw, grasping or locking feature on any embodiment of a connecting member.

FIGS. 1-6 illustrate that the spacer device 50 can be secured to the stabilization device 10 and in particular to a fixation rod or other similar elongate element 12. These embodiments illustrate how the adjacent level device 5 can be secured to the spine of the patient and are not intended to limit the scope of the invention. The spacer device 50 can be attached in different manners. For example, the spacer device 50 can be coupled with a pedicle screw fixed to a vertebra (similar to the fastener assembly 14 shown in FIG. 1). The spacer device 50 can be fixed to or placed adjacent to other suitable bony landmarks at an adjacent level such as, for example, a vertebral body, a pedicle, a spinous or transverse process or a lamina. In certain embodiments, the spacer device 50 can be tethered to suitable locations. For example, in one embodiment the spacer 60 is tethered to the stabilization device 10 by biocompatible flexible fibers such as, for example, natural or artificial ligaments. The spacer 60 also could be tethered to a spinal segment, including the spinal segment being treated at the primary surgical site or an adjacent spinal segment. Many variations are possible.

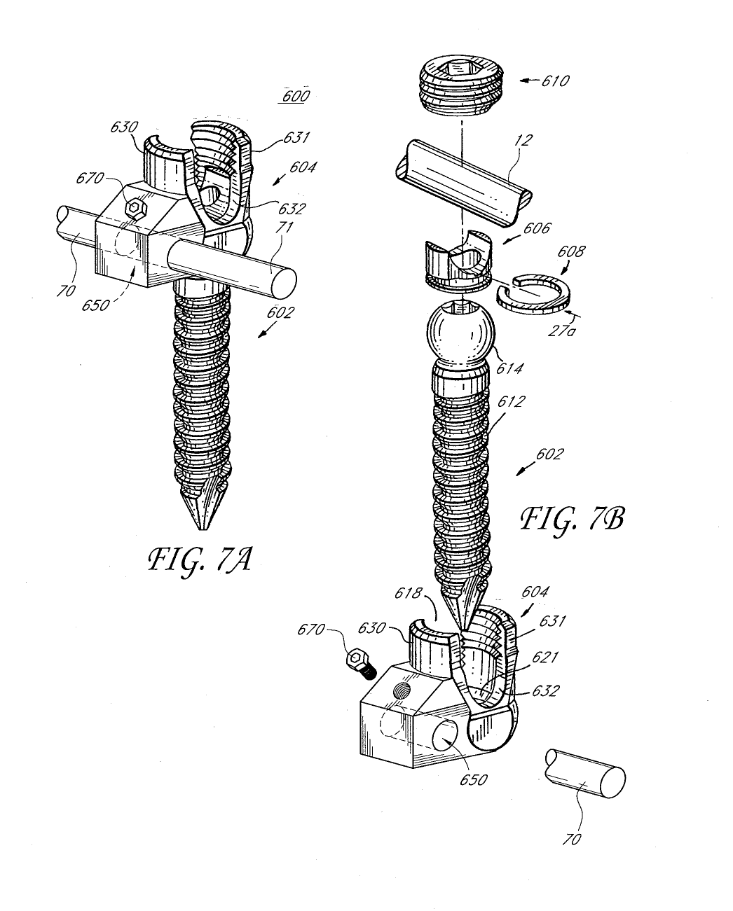

FIGS. 7A and 7B illustrate an embodiment of a connecting member 600 which can also couple one or more vertebrae and a spacer device, a stabilization device, or both. In one embodiment the connecting member 600 attaches one or more vertebrae to an elongate element 12, a spacer rod 70, or both. In some embodiments the connecting member 600 comprises or is connected to a screw or other fastening device to attach the connection member 100 to a vertebra. In one embodiment the connecting member 600 functions as a fastener assembly 14 for attaching a spacer device or a stabilization device to the spine. The connecting member 600 can have a hole or slot through which a screw can be inserted into bone. In various embodiments, the connecting member 600 can be configured to receive and securely couple with any of the embodiments of the spacer rod 70A-70H shown in FIGS. 1A-1H, including a snap fit or clamp.

The connecting member 600 can include one or more devices, e.g., screws such as a clamping screw or other threaded members, adapted to engage and secure one or more spacer rods 70 and/or one or more elongate elements 12. The clamping screws can be oriented in any direction. During implantation, the spacer rod 70 and/or the elongate element 12 can be slid through passageways in the connecting member 600 to adjust and position the spacer device relative to the stabilization device or relative to the spine of the patient. When the spacer device is in a suitable location, the clamping screw (and/or other threaded member or device) is secured to clamp the spacer device into position. The connecting member 600 can comprise any suitable substantially rigid material. For example, the connecting member 600 can comprise a metal such as titanium and its alloys, Nitinol, or a polymeric compound, including PEEK.

As illustrated in FIGS. 7A and 7B, one embodiment of a connecting member 600 preferably includes a screw portion 602, a housing 604, a passageway in the housing 650, a spacer member 606, a biasing member 608, and one or more clamping members, such as a cap screw 610 or a clamping screw 670. The screw portion 602 has a distal threaded portion 612 and a proximal, substantially spherical joint portion 614. The threaded portion 612 is inserted into a hole that extends away from a bone entry point into the vertebrae, as will be described below. The substantially spherical joint portion 614 is received in a substantially annular, partly spherical recess in the housing 604 in a ball and socket joint relationship. The spacer member 606, biasing member 608, and passageway in the housing 650 can each be adapted to receive or to engage either a spacer rod 70 or an elongate element 12.

As illustrated in FIG. 7B, the embodiment of the connecting member 600 is assembled by inserting the screw portion 602 into a bore in a passage 618 in the housing 604 until the joint portion 614 engages the annular recess. The screw portion 602 is retained in the housing 604 by the spacer member 606 and by the biasing member 608. The biasing member 608 provides a biasing force to drive the spacer member 606 into frictional engagement with the joint portion 614 of the screw member 602 and the annular recess of the housing 604. The biasing provided by the biasing member 602 frictionally maintains the relative positions of the housing 604 with respect to the screw portion 602. The biasing member 608 preferably is selected such that biasing force prevents unrestricted movement of the housing 604 relative to the screw portion 602. However, in some embodiments the biasing force is insufficient to resist the application of force by a physician to move the housing 604 relative to the screw portion 602. In other words, this biasing force is strong enough maintain the housing 604 stationary relative to the screw portion 602, but this force may be overcome by the physician to reorient the housing 604 with respect to the screw member 602. The proximal portion of the housing 604 includes a pair of upright members 630 and 631 that are separated by substantially “U”-shaped grooves 632. In various embodiments, a recess is adapted to receive or to engage or to house either the spacer rod 70 and/or an elongate element 12. In one embodiment, a recess for receiving an elongated element 12 is defined by the pair of grooves 632 between upright members 630 and 631. Elongated element 12 preferably is configured to be placed distally into the housing 604 in an orientation substantially transverse to the longitudinal axis of the housing 604.

In various embodiments, a passageway in the housing 650 is adapted to receive or to engage or to house at least one of the spacer rod 70 and an elongate element 12. In one embodiment, the passageway in the housing 650 is adapted to receive or to engage or to house a spacer rod 70 with a first end portion 71. The first end portion 71 can be slid through the passageway in the housing 650 and clamped into place by one or more clamping members, such as a clamping screw 670.

Additional features of a device similar to the connecting member 600 are also described in U.S. patent application Ser. No. 10/075,668, filed Feb. 13, 2002, published as U.S. Application Publication No. 2003/0153911A1 on Aug. 14, 2003 and issued as U.S. Pat. No. 7,066,937 on Jun. 27, 2006, and application Ser. No. 10/087,489, filed Mar. 1, 2002, published as U.S. Application Publication No. 2003/0167058A1 on Sep. 4, 2003 and issued as U.S. Pat. No. 6,837,889 on Jan. 4, 2005, and U.S. patent application Ser. No. 10/483,605, filed Jan. 13, 2004, published as U.S. Application Publication No. US 2004-0176766 on Sep. 4, 2003 and issued as U.S. Pat. No. 7,144,396 on Dec. 5, 2006, which are incorporated by reference in their entireties herein.

Although many of the embodiments described thus far have been illustrated with stabilization devices 10 that in certain cases relate to a fixation device with fastening assemblies attachable to the vertebral body or pedicles, other embodiments of a stabilization device 10 include facet joint fixation devices such as facet screws using a transfacet or translaminal approach or angle for insertion of the facet screws into vertebrae. In one embodiment, an adjacent level device 5 comprises at least one transfacet screw and any of the spacer device 58 described herein. The facet screw can be configured for a transfacet or translaminal approach and can be inserted through a hole or slot in a spacer rod of a spacer device. In another embodiment, a spacer device can comprise a connecting member (similar to connecting member 30, 100, 200, 300, 400, 500 or 600) that is configured to be coupled with a facet screw in a transfacet or translaminal approach to the spine.

B. Methods for Treating Adjacent Level Disc Disease

The adjacent level devices described above can be implanted during a surgical procedure, which advantageously can be a minimally invasive surgical procedure. In some embodiments, at least a portion of the adjacent level device 5 can be inserted through a cannula or access device. In other embodiments, at least a portion of the adjacent level device 5 is implanted through a minimally invasive access device, such as one that can be expanded at least at the distal end. Additional details on some minimally invasive apparatuses and methods suitable for use with the adjacent level device 5 are disclosed in U.S. patent application Ser. No. 10/693,250, filed Oct. 24, 2003, entitled “Methods and Apparatuses for Treating the Spine Through an Access Device,” and in U.S. application Ser. No. 11/241,811, filed Sep. 30, 2005, and in U.S. application Ser. No. 10/972,987, filed Oct. 25, 2004, which are hereby incorporated by reference herein in their entireties and made part of this specification.

The adjacent level device 5 and similar structures can also be applied to a patient using open surgical and mini-open surgical techniques. For example, in certain embodiments the adjacent level device 5 is implanted through a generally open surgery. With open surgery, the device 5 can be installed by attaching a stabilization device to a portion of the spine, cutting, piercing, or otherwise providing a passage through the interspinous ligament, inserting a spacer device, such as any of the spacer devices described herein between the spinous processes of an adjacent level (e.g., immediately above or below the spinous process of one of the fixed vertebrae), and attaching the spacer device to the stabilization device 10.

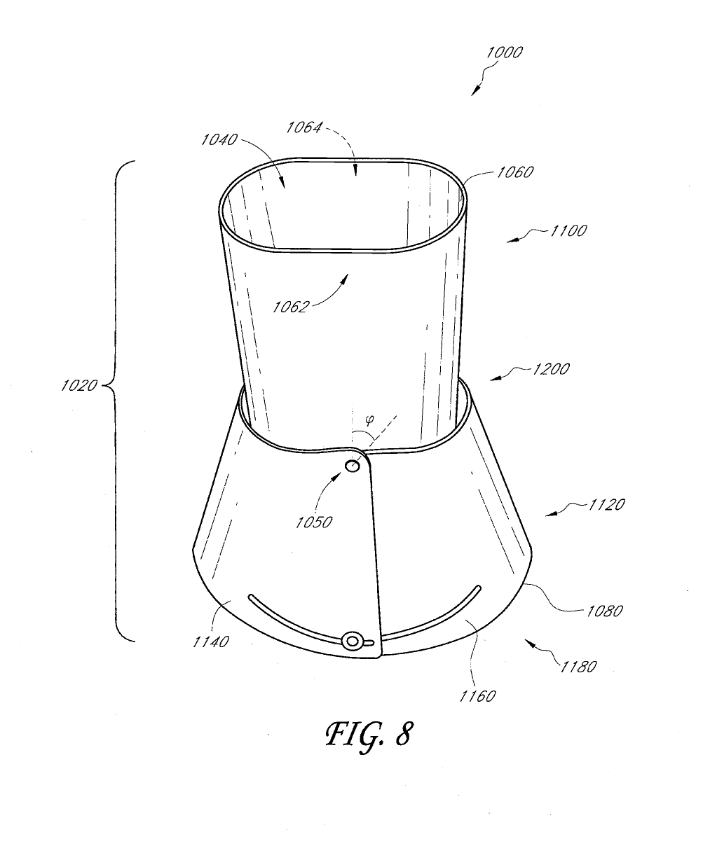

FIGS. 8 through 14 illustrate a wide variety of apparatuses and methods can be used to reduce adjacent level disc disease of the spine of a patient. For example, an access device can be used to access the vertebral space. The term “access device” is used in its ordinary sense to mean a device that can provide access and is a broad term and it includes structures having an elongated dimension and defining a passage, e.g., a cannula or a conduit. The access device is configured to be inserted through the skin of the patient to provide access during a surgical procedure to a surgical location within a patient, e.g., a spinal location. The term “surgical location” is used in its ordinary sense to mean a location where a surgical procedure is performed and is a broad term and it includes locations subject to or affected by a surgery. The term “spinal location” is used in its ordinary sense to mean a location at or near a spine and is a broad term and it includes locations adjacent to or associated with a spine that can be sites for surgical spinal procedures. The access device also can retract tissue to provide greater access to the surgical location. The term “retractor” is used in its ordinary sense to mean a device that can displace tissue and is a broad term and it includes structures having an elongated dimension and defining a passage, e.g., a cannula or a conduit, to retract tissue. Some retractors include blades to retract otherwise naturally continuous tissues between the skin and the spine to provide an access path to the spine.

Visualization of the surgical site can be achieved in any suitable manner, e.g., by direct visualization, or by use of a viewing element, such as an endoscope, a camera, loupes, a microscope, or any other suitable viewing element, or a combination of the foregoing. The term “viewing element” is used in its ordinary sense to mean a device useful for viewing and is a broad term and it also includes elements that enhance viewing, such as, for example, a light source or lighting element. In one embodiment, the viewing element provides a video signal representing images, such as images of the surgical site, to a monitor. The viewing element can be an endoscope and camera that captures images to be displayed on the monitor whereby the physician is able to view the surgical site as the procedure is being performed.

The systems are described herein in connection with minimally invasive postero-lateral and posterior spinal surgery. One such procedure is a two level postero-lateral fixation and fusion of the spine involving the L4, L5, and S1 vertebrae. In the drawings, such as FIGS. 1 and 9-15, the vertebrae will generally be denoted by reference letter V. The usefulness of the apparatuses and procedures is neither restricted to the postero-lateral or posterior approaches nor to the L4, L5, and S1 vertebrae. The apparatuses and procedures can be used in other anatomical approaches and with other vertebra(e) within the cervical, thoracic, lumbar, and sacral regions of the spine. The procedures can be directed toward surgery involving one or more vertebral levels. Some embodiments are useful for anterior and/or lateral procedures. A retroperitoneal approach can also be used with some embodiments. In one retroperitoneal approach, an initial transverse incision is made just left of the midline, just above the pubis, about 3 centimeters in length. The incision can be carried down through the subcutaneous tissues to the anterior rectus sheath, which is incised transversely and the rectus is retracted medially. At this level, the posterior sheath, where present, can be incised. With blunt finger dissection, the retroperitoneal space can be entered. The space can be enlarged with blunt dissection or with a retroperitoneal balloon dissector. The peritoneal sack can be retracted, e.g., by one of the access devices described herein.

It is believed that embodiments of the invention are also particularly useful where any body structures must be accessed beneath the skin and muscle tissue of the patient, and/or where it is desirable to provide sufficient space and visibility in order to manipulate surgical instruments and treat the underlying body structures. For example, certain features or instrumentation described herein are particularly useful for minimally invasive procedures, e.g., arthroscopic procedures. As discussed more fully below, one embodiment of an apparatus described herein provides an access device that is expandable, e.g., including an expandable distal portion. In addition to providing greater access to a surgical site than would be provided with a device having a constant cross-section from proximal to distal, the expandable distal portion prevents or substantially prevents the access device, or instruments extended therethrough to the surgical site, from dislodging or popping out of the operative site.

C. Systems and Devices for Establishing Access

In certain embodiments, retractors can be used to create an open space for accessing the spine. In one embodiment, the system includes an access device that provides an internal passage for surgical instruments to be inserted through the skin and muscle tissue of a patient to the surgical site. This access device can be a cannula or a series of cannulae. The access device can have a uniform cross section. The access device preferably has a wall portion defining a reduced profile, or low-profile, configuration for initial percutaneous insertion into the patient. This wall portion can have any suitable arrangement. In one embodiment, the wall portion has a generally tubular configuration that can be passed over a dilator that has been inserted into the patient to atraumatically enlarge an opening sufficiently large to receive the access device therein.

The wall portion of the access device preferably can be subsequently expanded to an enlarged configuration, by moving against the surrounding muscle tissue to at least partially define an enlarged surgical space in which the surgical procedures will be performed. In a sense, it acts as its own dilator. The access device can also be thought of as a retractor, and can be referred to herein as such. Both the distal and proximal portion can be expanded, as discussed further below. However, the distal portion preferably expands to a greater extent than the proximal portion, because the surgical procedures are to be performed at the surgical site, which is adjacent the distal portion when the access device is inserted into the patient. The surgical space provides a large working area for the surgeon inside the body within the confines of the cannula. Furthermore, the enlarged configuration provides a working area that is only as large as needed. As a result, the simultaneous use of a number of endoscopic surgical instruments, including but not limited to steerable instruments, shavers, dissectors, scissors, forceps, retractors, dilators, and video cameras, is made possible by the expandable access device.

While in the reduced profile configuration, the access device preferably defines a first unexpanded configuration. Thereafter, the access device can enlarge the surgical space defined thereby by engaging the tissue surrounding the access device and displacing the tissue outwardly as the access device expands. The access device preferably is sufficiently rigid to displace such tissue during the expansion thereof. The access device can be resiliently biased to expand from the reduced profile configuration to the enlarged configuration. In addition, the access device can also be manually expanded by an expander device with or without one or more surgical instruments inserted therein, as will be described below. The surgical site preferably is at least partially defined by the expanded access device itself. During expansion, the access device can move from a first overlapping configuration to a second overlapping configuration in some embodiments.

In some embodiments, the proximal and distal portions are separate components that can be coupled together in a suitable fashion. For example, the distal end portion of the access device can be configured for relative movement with respect to the proximal end portion in order to allow the physician to position the distal end portion at a desired location. This relative movement also provides the advantage that the proximal portion of the access device nearest the physician can remain substantially stable during such distal movement. In one embodiment, the distal portion is a separate component that is pivotally or movably coupled to the proximal portion. In another embodiment, the distal portion is flexible or resilient in order to permit such relative movement.

With reference to FIG. 8 in particular, an embodiment of an access device 1000 comprises an elongate body 1020 defining a passage 1040 and having a proximal end 1060 and a distal end 1080. The elongate body 1020 has a proximal portion 1100 and a distal portion 1120. In one embodiment, the proximal portion 1100 has an oblong or generally oval shaped cross section. The term “oblong” is used in its ordinary sense (i.e., having an elongated form) and is a broad term and it includes a structure having a dimension, especially one of two perpendicular dimensions, such as, for example, width or length, that is greater than another and includes shapes such as rectangles, ovals, ellipses, triangles, diamonds, trapezoids, parabolas, and other elongated shapes having straight or curved sides. The term “oval” is used in its ordinary sense (i.e., egg like or elliptical) and is a broad term and includes oblong shapes having curved portions. In other embodiments, the proximal portion 1100 can have a generally circular cross section.

Preferably, the proximal portion 1100 is sized to provide sufficient space for inserting multiple surgical instruments through the elongate body 1020 to the surgical location. The distal portion 1120 preferably is expandable and comprises first and second overlapping skirt members 1140, 1160. The degree of expansion of the distal portion 1120 is determined by an amount of overlap between the first skirt member 1140 and the second skirt member 1160 in one embodiment. The elongate body 1020 of the access device 1000 has a first location 1180 distal of a second location 1200. The elongate body 1020 preferably is capable of having a configuration when inserted within the patient wherein the cross-sectional area of the passage 1040 at the first location 1180 is greater than the cross-sectional area of the passage 1040 at the second location 1200.

The proximal portion 1100 is coupled with the distal portion 1120, e.g., with one or more couplers 1050. The proximal and distal portions 1100, 1120 are coupled on a first lateral side 1062 and on a second lateral side 1064 with the couplers 1050 in one embodiment. When applied to a patient in a postero-lateral procedure, either of the first or second lateral sides 1062, 1064 can be a medial side of the access device 1000, i.e., can be the side nearest to the patient's spine. The couplers 1050 can be any suitable coupling devices, such as, for example, rivet attachments. In one embodiment, the couplers 1050 are located on a central transverse plane of the access device 1000. The couplers 1050 preferably allow for at least one of rotation and pivotal movement of the proximal portion 1100 relative the distal portion 1120. The proximal portion 1100 is seen at an angle alpha α of about 20 degrees with respect to a transverse plane extending vertically through the couplers. One skilled in the art will appreciate that rotating or pivoting the proximal portion 1100 to the angle alpha α permits enhanced visualization of and access to a different portion of the spinal location accessible through the access device 1000 than would be visualized and accessible at a different angle. Depending on the size of the distal portion 1120, the angle alpha α can be greater than, or less than, 20 degrees. Preferably, the angle alpha α is between about 10 and about 40 degrees. The pivotable proximal portion 1100 allows for better access to the surgical location and increased control of surgical instruments.

In one embodiment, the access device has a uniform, generally oblong shaped cross section and is sized or configured to approach, dock on, or provide access to, anatomical structures. The access device preferably is configured to approach the spine from a posterior position or from a postero-lateral position. A distal portion of the access device can be configured to dock on, or provide access to, posterior portions of the spine for performing spinal procedures, such as, for example, fixation, fusion, or any other suitable procedure. In one embodiment, the distal portion of the access device has a uniform, generally oblong shaped cross section and is configured to dock on, or provide access to, generally posterior spinal structures. Generally posterior spinal structures can include, for example, one or more of the transverse process, the superior articular process, the inferior articular process, and the spinous process. In some embodiments, the access device can have a contoured distal end to facilitate docking on one or more of the posterior spinal structures. Accordingly, in one embodiment, the access device has a uniform, generally oblong shaped cross section with a distal end sized, configured, or contoured to approach, dock on, or provide access to, spinal structures from a posterior or postero-lateral position.

Further details and features pertaining to access devices and systems are described in U.S. Pat. No. 6,800,084, issued Oct. 5, 2004, U.S. Pat. No. 6,652,553, issued Nov. 25, 2003, application Ser. No. 10/678,744 filed Oct. 2, 2003, published as Publication No. 2005/0075540 on Apr. 7, 2005, which are incorporated by reference in their entireties herein.

D. Methods for Implanting an Apparatus to treat Adjacent Level Disc Disease

A type of procedure that can be performed by way of the systems and apparatuses described herein involves the placement of a device that treats, e.g., by reducing the likelihood of adjacent level degenerative disc disease while preserving or restoring a degree of normal motion after recovery. Such a procedure can be applied to a patient suffering degenerative disc disease or otherwise suffering from disc degeneration. A variety of adjacent level spinal implants that can be applied are described below. The access devices and systems described herein enable these devices and methods associated therewith to be practiced minimally invasively. A doctor can create one or more incisions through the skin of the back of a patient in order to insert an access device through the skin and tissue between the skin and the spine, providing a closed channel for delivering and affixing a device or implant to the spine.

In one embodiment an adjacent level device 5 is an implant comprising a stabilization device 10 (among various embodiments described herein) and a spacer device 50 (among various embodiments described herein). By way of illustration, embodiments of the stabilization device 10 can be used to treat, fix or assist in fusion of a first vertebra V1 and a second vertebra V2. The spacer device 50 can be used at one or more adjacent levels, such as between second vertebra V2 and a third vertebra V3 or between first vertebra V1 and a “zero” vertebra V0. Alternatively, the spacer device 50 can be used at a separate level that is not immediately adjacent to the primary treatment site with the stabilization device 10, such as a location that is two, three, or more vertebrae away from the primary treatment site. In some embodiments, the stabilization assembly 10 can be implanted in one procedure while the spacer device 50 can be implanted before, at the same time as, or in a subsequent procedure from the stabilization device 10. In some embodiments the spacer device 50 is advantageously installed in the same procedure as a stabilization device 10. In other embodiments, the spacer device 50 can be installed with, e.g., attached to, a pre-placed fixation assembly using a connecting member. For the purposes of illustrating the steps in a method of implanting an adjacent level device 5 comprising a stabilization device 10 and a spacer device 50, the following description will list steps in placing both types of devices in the body of the patient during one minimally invasive surgical procedure. The method is not limited to the order of steps set forth below, nor does it always require all steps or exclude other steps.

In one embodiment of a method for implanting the adjacent level device 5, after the doctor has created an incision through the skin and placed an access device through the skin to access the spine of the patient, a fastener assembly 14 including pedicle screws is implanted into each of the vertebrae V1 and V2. In some embodiments, the stabilization device 10 is mounted to bone by the screws in an early stage of a procedure, while in others the stabilization device is mounted in a later step. In some embodiments, a second set of screws and a second stabilization device 10 is mounted on another part of vertebrae V1 and V2. A spacer rod 70 is advanced through the spinous process ligament. For example, a surgical instrument can be used to form a passage through the ligament or other tissue located between adjacent spinous processes. In another embodiment, a portion of the device 5 can be configured to form such a passage. For example, as discussed above, the spacer rod 70 can have a sharp end to pierce the ligament. A spacer 60 can be inserted over the spacer rod 70. In certain embodiments, the elongate element 12 of the stabilization device 10 can be inserted into the patient. The spacer rod 70 can be coupled to the elongate element 12 by, for example, a ball and socket joint 30 (FIG. 2), any of the connecting members 100, 200, 300, 400, 500 or 600 (FIGS. 3-7), or in any other suitable manner. The spacer rod 70 and/or the elongate element 12 can be secured or clamped together by tightening screws, such as any of the variety of clamping screw or the set screw configurations described herein. Additional spacer devices 50 can be implanted in a similar manner, e.g., at the opposite end of a stabilization device 10 or at a next adjacent spinal segment on the same side of the stabilization device 10 as the initial spacer device 50.

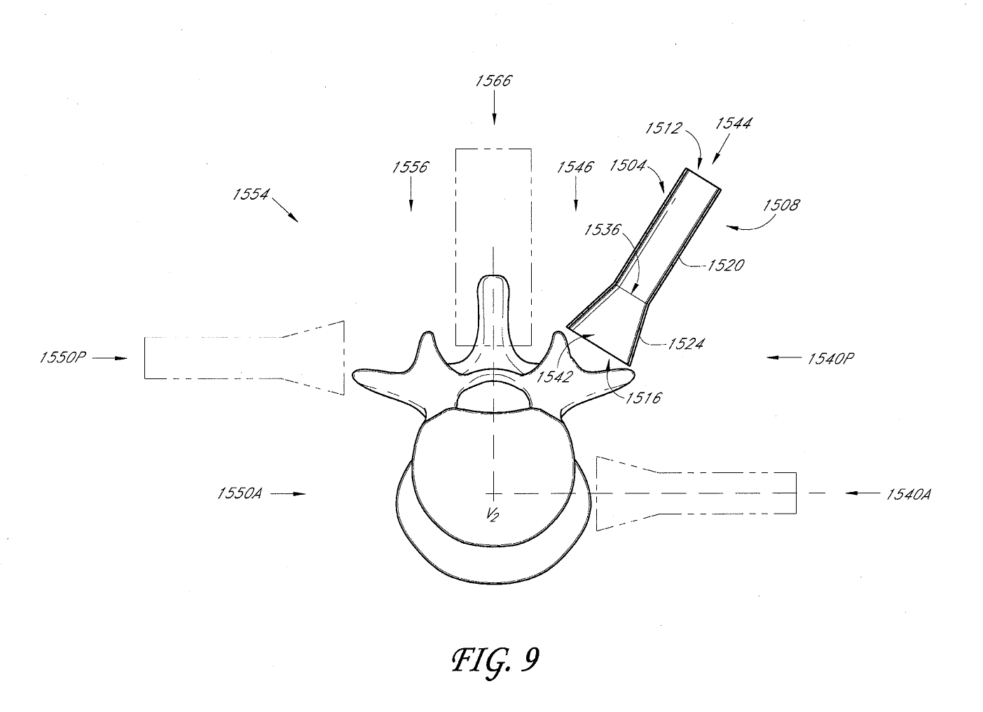

FIGS. 9-14 more particularly illustrate methods whereby an adjacent level device 5 can be delivered through an access device 1504. In an embodiment, access device 1504 is similar to the access device 1000 described above. The adjacent level device 5 can be delivered through the access device 1504 and implanted in a first intervertebral region I1, which is located at least partially between V1 and V2. An interspinous process space (ISPS) is at least partially located between adjacent interspinous processes. A first interspinous process space ISPS-1 is located within intervertebral region I1. Where provided, the spacer device 50 can be delivered through the access device 1504 and implanted in an intervertebral region I2, which is located at least partially between V2 and V3. A second interspinous process space ISPS-2, which is located within intervertebral region I2. The stabilization device 10 can be any suitable fixation, fusion, stabilization, dynamic stabilization, or other type of implant, e.g., any of the variety of embodiments described herein. The spacer device 50 can be any suitable implant, e.g., any of the embodiments described herein.

Referring to FIG. 9, in one method, access to the intervertebral regions I1 and/or I2 is provided by inserting the access device 1504 into the patient. The access device 1504 can be configured in a manner similar to any of the access devices disclosed herein, such as in one embodiment the access device 1000 discussed with FIG. 8, or in a manner similar to an expandable conduit and can be inserted in a similar manner, e.g., over a dilator. The access device 1504 preferably has an elongate body 1508 that has a proximal end 1512 and a distal end 1516. In one embodiment, the elongate body 1508 comprises a proximal portion 1520 and a distal portion 1524. The distal portion 1524 preferably is expandable to the configuration illustrated in FIGS. 9 through 14. At least one passage 1528 extends through the elongate body 1508 between the proximal end 1512 and the distal end 1516.

The elongate body 1508 has a length between the proximal end 1512 and the distal end 1516 that is selected such that when the access device 1504 is applied to a patient during a surgical procedure, the distal end 1516 can be positioned inside the patient adjacent a spinal location, and, when so applied, the proximal end 1512 preferably is located outside the patient at a suitable height. As discussed below, various methods can be performed through the access device 1504 by way of a variety of anatomical approaches, e.g., anterior, lateral, transforaminal, postero-lateral, and posterior approaches. The access device 1504 can be used for any of these approaches and can be particularly configured for any one of or for more than one of these approaches.

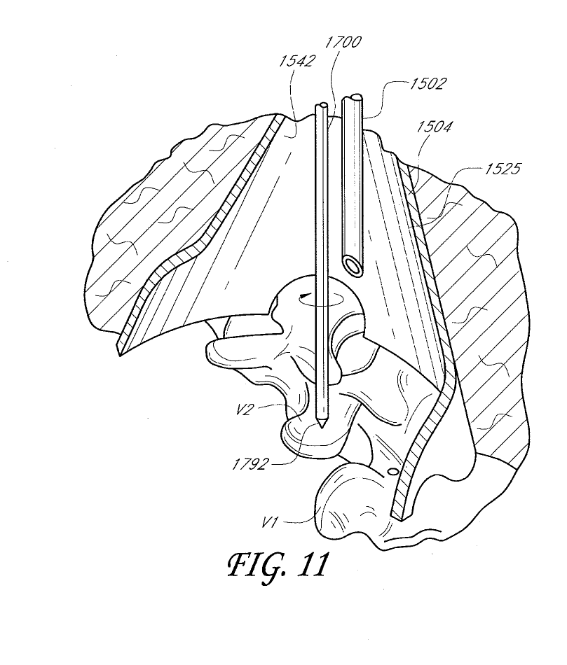

The access device 1504 can be configured to be coupled with a viewing element (not illustrated in FIG. 9, but see endoscope 1502 in FIGS. 11-14) in one embodiment. The distal portion 1524 of the access device 1504 has an aperture 1536 into which the viewing element can be inserted, such that a proximal portion of the viewing element lies external to the proximal portion 1520 and a distal portion of the viewing element lies within the distal portion 1524 of the access device 1504. The viewing element can be any suitable viewing element, such as an endoscope, a camera, loupes, a microscope, a lighting element, or a combination of the foregoing. The viewing element can be an endoscope or a camera which capture images to be displayed on a monitor. Further details of the access device 1504 are set forth in an application entitled “Minimally Invasive Access Device and Method,” filed Oct. 2, 2003, U.S. application Ser. No. 10/678,744, published as Publication No. 2005/0075540 on Apr. 7, 2005, which is hereby incorporated by reference in its entirety.

Various methods can be performed through the access device 1504 by way of a variety of anatomical approaches, e.g., anterior, lateral, transforaminal, postero-lateral, and posterior approaches, and the dashed-line outlines of various access devices, such as access device 1504, in FIG. 9. Although all these approaches are contemplated, only some of these approaches are discussed in detail and illustrated in the figures. In the illustrated methods, the distal end 1516 of the access device 1504 can be inserted postero-lateral, as indicated by an arrow 1544, to a surgical location adjacent to at least one vertebra and preferably adjacent to two vertebrae, e.g., the first vertebra V1 and the second vertebra V2, to provide access to at least a portion of the intervertebral region I1 or intervertebral region I2. In some embodiments, the access device 1504 is inserted to a surgical location adjacent to three vertebrae—V1, V2 and V3—to provide access to at least a portion of the intervertebral region I1 and intervertebral region I2. In another method, the access device 1504 is inserted laterally, as indicated by an arrow 1540A for a lateral approach near the vertebral body at an anterior side of the spine, and as indicated by an arrow 1540P for a lateral approach near the spinal processes at a posterior side of the spine. In another method, the access device 1504 is inserted posteriorly, as indicated by an arrow 1546 or the dashed outline of any access device shown with arrow 1566, to provide access to at least a portion of the intervertebral region I1, at least a portion of the intervertebral region I2, or at least a portion of the intervertebral region I1 and intervertebral region I2. In some embodiments the access device can have a constant cross-section, such as shown with the dashed outline at arrow 1566, which can be used to access the region between spinous processes for inserting a spacer, as will be described below. In other embodiments, an access device 1504 can access the spine at arrow 1566. As discussed above, the access device 1504 can have a first configuration for insertion to the surgical location over the intervertebral region I1 or intervertebral region I2 and a second configuration wherein increased access is provided to the intervertebral region I1 or intervertebral region I2. As discussed above, the access device 1504 can have a first configuration for insertion to the surgical location over the intervertebral regions I1 and I2 and a second configuration wherein increased access is provided to the intervertebral regions I1 and I2. The second configuration can provide a cross-sectional area at the distal end 1516 that is larger than that of the first configuration at the distal end 1516, similar to the access device 1000 described above.

In some methods of applying an adjacent level device 5 (not shown here), a second access device, such as an expandable conduit or other suitable access device, can be inserted into the patient. For example, a second access device could be inserted through a postero-lateral approach on the opposite side of the spine, as indicated by an arrow 1554, to provide access to at least a portion of an intervertebral region, e.g., the intervertebral region I. In another embodiment, a second access device could be inserted through a posterior approach on the opposite side of the spine, as indicated by an arrow 1556 to provide access to at least a portion of an intervertebral region, e.g., the intervertebral region I. This second access device can provide access to the intervertebral region I1 at about the same time as the first access device 1504 or during a later or earlier portion of a procedure. In one method, an implant is inserted from both sides of the spine using first and second access devices. Likewise, a second access device can be inserted as described herein to provide access to at least a portion of two intervertebral regions, e.g., the intervertebral regions I1 and I2.

In various applications, one or more adjacent level devices can be delivered through one or more access devices, such as the access device 1504, from different directions. For example, a first adjacent level device could be delivered through a first access device from the approach indicated by the arrow 1544, and a second adjacent level device could be delivered through a second access device from the approach indicated by the arrow 1554. In another method, a first portion of a first adjacent level device, e.g., a portion to be coupled with the superior vertebra defining the intervertebral region I, could be delivered through a first access device from the approach indicated by the arrow 1544, and a second portion of the first adjacent level device, e.g., a portion to be coupled with the inferior vertebra defining the intervertebral region I, could be delivered through a second access device from the approach indicated by the arrow 1556. Thus, any combination of single, multiple implants, or implant sub-components can be delivered through one or more access devices from any combination of one or more approaches, such as the approaches indicated by the arrows 1540A, 1540P, 1544, 1546, 1550A, 1550P, 1554, 1556, or any other suitable approach to either intervertebral region I1 or intervertebral region I2, or both intervertebral regions I1 and I2.

As discussed above, in some methods, suitable procedures can be performed to prepare the spine to receive an implant, e.g., the adjacent level device. For example, the surfaces of the vertebrae V1, V2 and V3 or any surface in the intervertebral region I1 or I2 can be prepared as needed, e.g., the surfaces can be scraped or scored, and/or holes can be formed in the vertebrae to receive one or more features formed on a surface of the adjacent level device. Also, in some procedures, degraded natural disc material can be removed in a suitable manner, e.g., a discectomy can be performed.

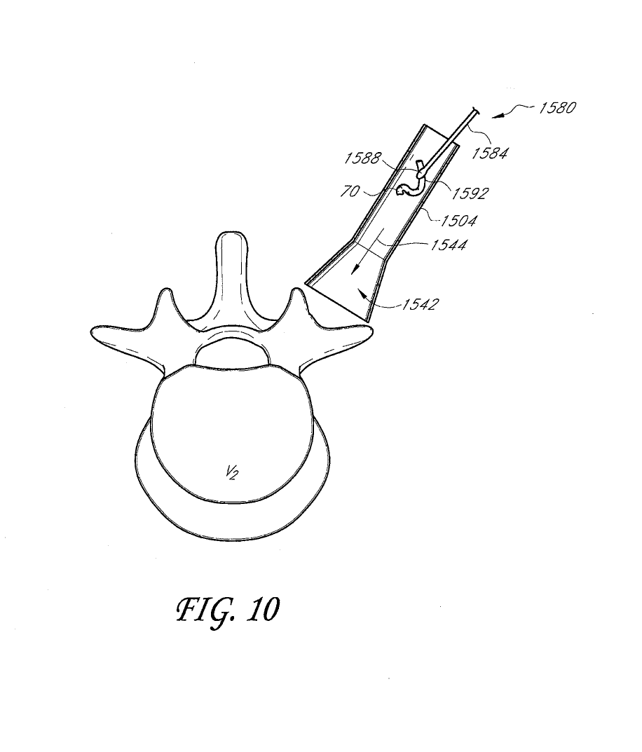

FIG. 10 illustrates a portion of an embodiment of a method of applying an adjacent level device or a portion of an adjacent level device through the access device 1504. In one embodiment, the device 5 is delivered through the access device 1504 in parts or sub-components to be assembled near the spine within the working space of the access device. As illustrated, FIG. 10 depicts the spacer rod 70 as discussed in any of its embodiments herein, being advanced through access device 1504 to a spine. In particular, after the access device 1504 is actuated to the expanded configuration, the adjacent level device is delivered postero-laterally as indicated by the arrow 544 to a surgical location defined by the distal end 1516 of the access device 1504 at one lateral side of the vertebrae V1, V2, and/or V3 and the intervertebral regions I and/or II. In one application, in order to facilitate insertion of the adjacent level device, visualization of the surgical site can be achieved in any suitable manner, e.g., by use of a viewing element (not shown), as discussed above.

In one procedure, a gripping apparatus 1580, is coupled with one or more portions and/or surfaces of the adjacent level device to facilitate insertion of the adjacent level device. In one embodiment, the gripping apparatus 1580 has an elongate body 1584 that extends between a proximal end (not shown) and a distal end 1588. The length of the elongate body 1584 is selected such that when the gripping apparatus 1580 is inserted through the access device 1504 to the surgical location, the proximal end extends proximally of the proximal end 1512 of the access device 1504. This arrangement permits the surgeon to manipulate the gripping apparatus 1580 proximally of the access device 1504. The gripping apparatus 1580 has a grip portion 1592 that is configured to engage the adjacent level device. In one embodiment, the grip portion 1592 comprises a clamping portion configured to firmly grasp opposing sides of the implant. The clamping portion can further comprise a release mechanism, which can be disposed at the proximal end of the gripping apparatus 1580, to loosen the clamping portion so that the adjacent level device can be released once delivered to the surgical location of the spine. In another embodiment, the grip portion 1592 comprises a jaw portion with protrusions disposed thereon, such that a portion of the adjacent level device fits within the jaw portion and engages the protrusions. In another embodiment, the grip portion 1592 comprises a malleable material that can conform to the shape of the adjacent level device and thereby engage it. Other means of coupling the gripping apparatus 1580 to the adjacent level device known to those of skill in the art could also be used, if configured to be inserted through the access device 1504. In one method of delivering the adjacent level device to the surgical location, the gripping apparatus 1580 is coupled with the adjacent level device, as described above. The gripping apparatus 1580 and the adjacent level device are advanced into the proximal end 1512 of the access device 1504, to the surgical space 1542, and further into the surgical space 1542.

In one embodiment an adjacent level device can be delivered to a surgical site in separate parts. In one embodiment, a stabilization device 50, which can be a fixation, fusion, stabilization or dynamic stabilization assembly, is implanted first. One procedure performable through the access device 1504, described in greater detail below, is a two-level spinal fixation using a stabilization device 50. Surgical instruments inserted into the expandable access device 1504 can be used for debridement and decortication. In particular, the soft tissue, such as fat and muscle, covering the vertebrae can be removed in order to allow the physician to visually identify the various “landmarks,” or vertebral structures, which enable the physician to locate the location for attaching a fastener, such a fastener assembly 14, discussed herein, or other procedures, as will be described herein. Allowing visual identification of the vertebral structures enables the physician to perform the procedure while viewing the surgical area through the endoscope, microscope, loupes, etc., or in a conventional, open manner. As illustrated, the end of an endoscope 1502 can be used to visualize the procedure within the access device 1504.

Tissue debridement and decortication of bone are completed using one or more debrider blades, bipolar sheath, high speed burr, and additional conventional manual instruments. The debrider blades are used to excise, remove and aspirate the soft tissue. The bipolar sheath is used to achieve hemostasis through spot and bulk tissue coagulation. The debrider blades and bipolar sheath are described in greater detail in U.S. Pat. No. 6,193,715, assigned to Medical Scientific, Inc., which is hereby incorporated by reference in its entirety herein. The high speed burr and conventional manual instruments are also used to continue to expose the structure of the vertebrae.

A subsequent stage is the attachment of fasteners to the vertebrae V. Prior to attachment of the fasteners, the location of the fastener attachment is confirmed. In the exemplary embodiment, the pedicle entry point of the L5 vertebrae is located using visual landmarks as well as lateral and A/P fluoroscopy, as is known in the art. With reference to FIG. 11, the entry point at a hole 1792 is prepared with an awl 1700. The hole 1792, in one embodiment a pedicle hole, is completed using instruments known in the art such as a straight bone probe, a tap, and a sounder. The sounder, as is known in the art, determines whether the hole that is made is surrounded by bone on all sides, and that there has been no perforation of the pedicle wall.

After a hole in the pedicle is provided at the entry point at a hole 1792 (or at any point during the procedure), an optional step is to adjust the location of the distal portion of the access device 1504. This can be performed by inserting an expander apparatus (not shown) into the access device 1504, expanding the distal portions 1524, and contacting the inner wall of the skirt portion 1525 to move the skirt portion 1525, to the desired location. This step can be performed while the endoscope is positioned within the access device 1504, and without substantially disturbing the location of the proximal portion of the access device 1504 to which an endoscope mount platform can be attached.

In one embodiment, a fastener assembly 14 can be inserted which is particularly applicable in a procedures involving fixation. A fastener assembly 14 is described in greater detail in U.S. patent application Ser. No. 10/075,668, filed Feb. 13, 2002 and application Ser. No. 10/087,489, filed Mar. 1, 2002, which are hereby incorporated by reference in their entirety. Fastener assembly 14 can include a screw, a screw portion, a housing, a spacer member, a biasing member, or a clamping member, such as a cap screw. The screw portion has a distal threaded portion and a proximal, substantially spherical joint portion. The threaded portion is inserted into the hole 1792 in the vertebrae, as will be described below. The substantially spherical joint portion is received in a substantially annular, part spherical recess in the housing in a ball and socket joint relationship. The fastener assembly 14 can be attached to the spine and to an elongated member 12 (or a fixation plate, fusion-assisting device, or stabilization rod) using any variety of tools appropriate for actuating or connecting the fastener assembly 14, such as a screwdriver or other tool. In certain embodiments, the fastener assembly 14 can be attached to a spacer device 50 or to a type of connecting member 30, 100, 200, 300, 400, 500 or 600 to connect the spacer device 50 to bone or some other component or device placed in the body.

For a two-level fixation, it can be necessary to prepare several holes and attach several fastener assemblies 14 on one or both sides of a spinous process. Typically, the access device 1504 will be sized in order to provide simultaneous access to all vertebrae in which the surgical procedure is being performed. In some cases, however, additional enlargement or repositioning of the distal portion of the expandable conduit can be required in order to have sufficient access to the outer vertebrae, e.g., the L4 and S1 vertebrae. The expander apparatus can be repeatedly inserted into the access device 1504 and expanded in order to further open or position the skirt portion 1525. In one procedure, additional fasteners are inserted in the L4 and S1 vertebrae in a similar fashion as the fastener assembly 14 is inserted in to the L5 vertebra as described above., (When discussed individually or collectively, a fastener assembly and/or its individual components will be referred to by the reference number, e.g., fastener assembly 14, where the fastener assembly 14 can have a housing or other attachment structure attached.)

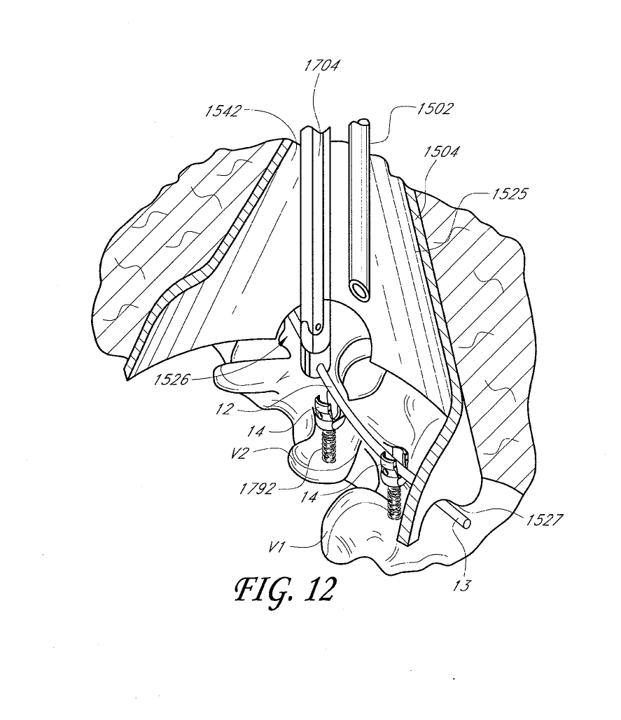

As illustrated in FIGS. 12-14, a grasper apparatus 1704 can be used to insert the elongated member 12 (or fixation plate, fusion-assisting device, or stabilization rod) into the surgical space 1542, or in one embodiment an operative space, defined at least partially by the skirt portion 1525 of the access device 1504. The cut-out portions 1526 and 1527 provided in the skirt portion 1525 assist in the process of installing the elongated member 12 with respect to the housings of the fastener assemblies 14. The cut-out portions 1526 and 1527 can be used to allow a second end portion 13 of the elongated member 12 to extend beyond the operative space without raising or repositioning the skirt portion 1525. The elongated member 12 is positioned within a recess in the housing of each fastener assembly 14. In one embodiment, the elongated member 12 is positioned in an orientation substantially transverse to the longitudinal axis of each housing of the fastener assembly 14. In some embodiments, the elongated member 12 can have a hole or slot though which a fastener assembly 14 is actuated into bone.

In some embodiments the cut-out portions 1526 and 1527 of the skirt portion 1525 also provide access within the skirt portion 1525 to an interspinous process space, such as ISPS-2, through which a spacer rod 70 or a spacer device 50 is inserted for placement of the spacer 60. In other embodiments, no cut-out portions are needed as the interspinous process space, such as ISPS-2, can be accessed through the open distal end of the skirt portion 1525 of the access device 1504.

In one embodiment, a tool such as a gripper, pliers, or a grasping apparatus 1704 can be inserted into the working space of the access device 1504 to bend or configure implants to conform to the boney geography of the patient in a manner appropriate for treatment of the spine. As illustrated in the embodiment depicted in FIG. 12, an elongate element 12 which is contiguous with a spacer rod 70 can be bent or directed through an intervertebral space 12 by a grasping apparatus 1704. This step can be taken prior to or subsequent to the connection of the elongate element 12 to one or more fastener assemblies 14. In some embodiments, the spacer rod 70 can be threaded or directed through an interspinous process space ISPS-2 via a pre-existing channel in the interspinous ligament, or via a channel created by inserting tools to make a channel in the interspinous ligament, or via a channel created by the spacer rod 70. As described above, certain embodiments of a spacer rod such as spacer rod 70A can have a sharp or conical point which can be used to thread through or to pierce tissue to access the intervertebral region I2.

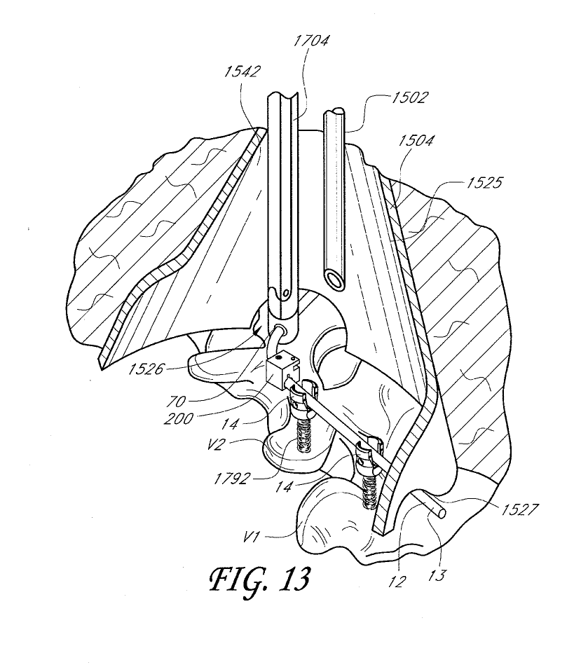

In several embodiments of an adjacent level device, the device comprises a stabilization device, such as stabilization device 10, a spacer device, such as spacer device 50, and a connecting member to connect the stabilization device 10 to the spacer device 50. Any variation of connecting member 30, 100, 200, 300, 400, 500 or 600 discussed herein can be installed, including connecting members with a hole, slot, or screw which can be used with a screw housing or in conjunction with a fastener assembly 14 as described above. As illustrated in FIG. 13, a connecting member 200 is used, but any type of connecting member as disclosed above may be used. In some embodiments, the connecting member can be slidable along an exposed end of a elongate element 12 and have a spacer rod 70 attached to it and appropriately locked in place with a screw, as described above. In other embodiments, a spacer rod 70 or elongate member 12 may be dropped into an open channel in the connecting member, such as an open lateral connecting member 500A as shown in FIG. 6A. In other embodiments, the connecting member can be secured to a spacer rod 70 such as a tether or an anchor point for a snap fit spacer rod, such as spacer rod 70E. In certain embodiments where the spacer rod has a sharp point (such as is illustrated with spacer rod 70A in FIG. 1A) a channel within the connecting member can be used to contain the sharp point to shield the point from tissue when installed. As illustrated, connecting element 200 can attach a spacer rod 70 to an elongate member 12.

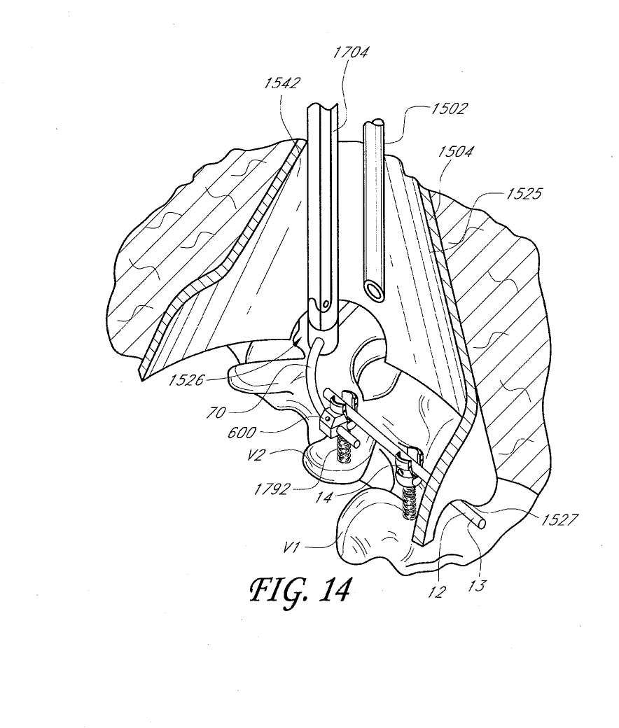

FIG. 14 illustrates an embodiment of a portion of a method of installing an adjacent level device comprising a connecting member 600 comprising a bone screw. It is similar in many ways to the embodiment described with FIG. 13, but instead of using two fastening assemblies 14 per elongate element 12, the connecting member 600 is used with one fastening assembly 14. Although not illustrated here, in one embodiment, the connecting member 600 may be used to attach multiple stabilization devices 10 (which may be aligned in different orientations or collinear) to one or more spacer devices 50. In one embodiment, connecting members 600 may be used in place of any fastening assembly 14. In one embodiment, two spacer devices 50 may be connected to a stabilization device 10 by more than one connecting member 600.

Placement of the spacer 60 in an intervertebral region I2 can be accomplished in a number of methods. The spacer 60 can be placed in an interspinous process space ISPS-2. In one embodiment, the spacer 60 can be placed in a facet joint. Each of these locations has a ligament or disk-type structure which would need to be pierced or severed to make room for the spacer.

In open procedures, the spacer 60 can be placed in a channel of a ligament created by any tool. The ligament can have a hole pierced or drilled in it, or the ligament can be cut open for placement of the spacer 60. In less invasive procedures, including minimally invasive procedures using an access device, cannula, or expandable access device as described above, the spacer 60 or the spacer rod 70 can be threaded through a ligament using the blunt or sharpened leading end of the spacer 60 or spacer rod 70 to pierce or tear through the ligament. (See embodiments of spacer rod 70A in FIG. 1A and spacer 60H in FIG. 1H) The spacer 60 or spacer rod 70 can be “hooked” through a ligament. In certain embodiments, the ligament can be access via an oblique approach or via a lateral approach. In some embodiments, the ligament can be pierced or cut open with an incision using separate tools through the access device. Retractors can be used in and around ISPS-2. In one embodiment, the spacer 60 or spacer rod 70 can advanced by a stab incision using a tool from one or two sides of the spine. A lateral stab incision can be used to thread or advance the spacer 60 or spacer rod 70 through the ligament. A percutaneous device can be used to create a hole, channel or incision through a ligament. A percutaneous device can be used to advance a spacer 60 or spacer rod 70 though a portion of a intervertebral region I2. Once a channel, hole or incision in the ligament is established, the spacer rod 70 can be advanced through the ligament and a spacer 60 can be advanced along the spacer rod 70 to the position in I2 or ISPS-2 as discussed above. In one embodiment, an open end of a spacer rod 70 is accessible after the rod 70 traverses the ligament and a spacer 60 can be placed over the open end of the spacer rod 70 and then advanced to the target location. In other embodiments, the spacer 60 can already be slidably attached to the spacer rod 70 and advanced to the target location. In one embodiment, once the spacer rod 70 is in the appropriate target location to allow placement of the spacer 60 in the target location, the connecting member 100 can be attached or locked to the spacer rod 70. In other embodiments using a flexible spacer rod 70, the spacer rod 70 can be locked to the connecting member or fastener assembly 14 prior to placement. In one embodiment, a spacer 60 may be inserted through a ligament with a spacer rod 70 already attached to the spacer 60, such as in one embodiment, a sharpened spacer 60H with a flexible spacer rod 1C.

The order of the steps as described above can be transposed or accomplished in alternative sequences. For instance, a spacer device 50 can be inserted in an interspinous ligament prior to the installation of an elongate element 12, or vice versa. Several combinations of steps are possible.

Although many of the embodiments of methods of installing an adjacent level device described thus far have been illustrated with stabilization devices 10 that in certain cases relate to a fixation device with fastening assemblies attachable to the vertebral body or pedicles, other embodiments of a stabilization device 10 include facet joint fixation devices such as facet screws using a transfacet or translaminal approach or angle for insertion of the facet screws into bone. In one embodiment, an adjacent level device comprises a spacer device 58 (not illustrated) which comprises a spacer 60 and a spacer rod 70 which can be a spacer elongate element or a spacer plate that is configured to be attached to one or more facet screws. The spacer elongate element or spacer plate can have a hole or slot through which the facet screw can be inserted into bone. The facet screw can be configured for a transfacet or translaminal approach and is placed through a hole or slot in a spacer rod 70 of a spacer device 50 (or any embodiment of the spacer devices disclosed herein). The spacer rod 70 and spacer 60 can be inserted through a ligament in ISPS-2 in a manner as described above, after which one or more facet screws can be inserted through the spacer rod 70 and into the facet joint through either a transfacet or translaminal approach. In another embodiment, a spacer device 50 can comprise a connecting member (similar to connecting member 30, 100, 200, 300, 400, 500 or 600) which is configured to be attached to a facet screw in a transfacet or translaminal approach to the spine. In steps in inserting a spacer device 50 with a connecting member can use the steps described above for inserting and positioning a spacer 60 or spacer rod 70 first, inserting the spacer rod 70 into a connecting member, then inserting the facet screws through a hole or slot in the connecting member in a transfacet or translaminal approach to the spine. In another embodiment, the connecting member is attached to bone near the facet joints by the insertion of the facet screws prior to the placement of a spacer rod 70 or spacer 60 into the ligament or the ISPS-2 as discussed in any of the variety of steps disclosed above, with the spacer rod 70 and connecting member being attached in a subsequent step.

Another procedure that can be performed through the access device 1504 involves treatment or replacement of one or more joints. Some patients who are suffering from degenerative disc disease can also suffer from degenerative facet joint disease. While treatment or replacement of both a disc and a facet joint in such a patient is possible during the same operation using other methods, such an operation would be very complicated because it would likely require that the spine be approached both anteriorly and posteriorly. In contrast, in some approaches described hereinabove, the access device 1504 would provide sufficient access to spine to facilitate treatment of a part of the spine with the adjacent level device, including to one or more disc or facet joints to facilitate treatment or replacement of one or more facet joints. For example, the postero-lateral approaches indicated by the arrows 1544, 1554 in FIG. 9 could provide access to a disc in the intervertebral region I1 and an adjacent facet joint. In another method, first and second access devices could be applied in any combination of the lateral, posterior and postero-lateral approaches indicated by the arrows 1540A, 1540P, 1550A, 1150P, 1546, 1556, 1544, and 1554, or other approach, to provide access to an intervertebral region I1 and an adjacent facet joint. In one method three or more joints are replaced, e.g., a disc in the intervertebral region I1 and the two corresponding, adjacent facet joints by way of one or more access device applied along any combination of the approaches 1540A, 1540P, 1550A, 1550P, 1546, 1556, 1544, and 1554, or other approach.

E. Methods for Removing an Apparatus to treat Adjacent Level Disc Disease

Referring back to FIG. 10, although the methods discussed above are particularly directed to the insertion of an adjacent level adjacent level device, the access device 1504 can also be used advantageously to remove the adjacent level adjacent level device. It can be desirable to remove the adjacent level device if the patient's spine condition changes or if the performance of the adjacent level device is compromised, e.g., through wear or subsidence (reduction in the height of the spacer), or if the adjacent level degenerative disease has advanced to the adjacent level and more stabilization is required at that level. In one application, the gripping apparatus 1580 can also be further configured to facilitate removal as well as insertion. By providing minimally invasive access to the surgical space 1542, the access device 1504 can be used analogously as described above with reference to the removal of a previously inserted adjacent level adjacent level device. Upon removal of the adjacent level device or portions thereof, various subsequent procedures can be performed in the surgical space 1542. For example, a new adjacent level adjacent level device can be inserted through the access device 1504 into the surgical space 1542. In some embodiments, only a portion or subassembly of the adjacent level adjacent level device need be replaced. In an embodiment in which only the spacer device 50 need be replaced or removed, the connecting member (any of connecting member 30, 100, 200, 300, 400, 500 or 600 as described above) is actuated to release the spacer rod 70. In certain embodiments, the spacer 60 can be slid off or cut away from the spacer rod 70. A replacement spacer 60 can be used to replace the removed spacer 60. Alternatively, an entire spacer device 50 can be replaced. In certain embodiments, the connector member can be replaced as well. In other embodiments, the initial spacer device 50 is removed from the initial adjacent level, such as at ISPS-2, and an additional stabilization device 10 is implanted at the intervertebral region I2. In some embodiments, a new spacer device 50 can be implanted at another level of the spine, such as at intervertebral region I3, which is includes at least a portion of vertebra V2 and vertebra V3 as well as the space between the vertebrae. Other procedures that could be performed after removing the previously inserted adjacent level adjacent level device include the insertion of a fusion device where it is determined that fusion is a more suitable treatment than dynamic stabilization or the placement of adjacent level spacers. Such a determination can arise from a change in the condition of the spine, e.g., due to the onset of osteoporosis, that makes additional use of an adjacent level device at that intervertebral region inappropriate.

The foregoing methods and apparatuses advantageously provide minimally invasive treatment of spine conditions in a manner that preserves some degree of motion between the vertebrae on either side of the replaced disc. Accordingly, trauma to the patient can be reduced, thereby shortening recovery time. Many of the implants provide a more normal post-recovery range of motion of the spine, which can reduce the need for additional procedures.

It will be understood that the foregoing is only illustrative of the principles of the invention, and that various modifications, alterations, and combinations can be made by those skilled in the art without departing from the scope and spirit of the invention. Accordingly, it is not intended that the invention be limited, except as by the appended claims.

What is claimed is:

1. A spinal stabilization apparatus, comprising:
a primary stabilization device comprising:
a first screw configured to be inserted into a first vertebra;
a second screw configured to be inserted into a second vertebra;
a first elongate member extendable between the first and second screws, the first elongate member configured to reduce at least some of the range of motion of the first and second vertebrae;
a device configured to intermittently interact with an adjacent spinal level, comprising:
a spacer configured to be inserted between a spinous process of the first vertebra and a second vertebra adjacent to the first vertebra; and
a second elongate member configured to interconnect the spacer and the primary stabilization device.

2. The spinal stabilization device of claim 1, wherein the first elongate member is a rigid rod.

3. The spinal stabilization device of claim 1, wherein the first elongate member is a flexible member.

4. The spinal stabilization device of claim 1, wherein the spacer is configured to occupy up to one-half of the normal separation between the spinous processes between which it is inserted.

5. The spinal stabilization device of claim 1, wherein the spacer is a compressible member.

6. The spinal stabilization device of claim 1, wherein the second elongate member is rigid.

7. The spinal stabilization device of claim 1, wherein the second elongate member is flexible.

8. The spinal stabilization device of claim 1, wherein the second elongate member comprises a sharp end capable of creating a passage through paraspinal tissue.

9. An apparatus for reducing adjacent level disc disease, comprising:
a fixation device comprising:
a first screw configured to be inserted into a first vertebra;
a second screw configured to be inserted into a second vertebra;
a device configured to reduce adjacent level disc disease, comprising:
a spacer configured to be inserted between a spinous process of the second vertebra and a third vertebra adjacent to the second vertebra; and
an elongate member configured to interconnect the spacer and the fixation device.

10. The apparatus of claim 9, further comprising a stabilization member, wherein the first screw is configured to be inserted through a pedicle of the first vertebra and the second screw is configured to be inserted into a pedicle of the second vertebra; and wherein the first and second screws are configured to receive and securely connect to the stabilization member.

11. The apparatus of claim 10, wherein the stabilization member is a rigid rod.

12. The apparatus of claim 10, wherein the stabilization member is a flexible member configured to preserve at least some of the normal range of motion of the first and second vertebrae.

13. The apparatus of claim 9, wherein the elongate member is a rigid rod.

14. The apparatus of claim 9, further comprising a connecting member having a passage and a clamping device, the connecting member configured to be coupled with at least one of the screws, the passage configured to receive the elongate member, and the clamping device configured to clamp the elongate member in the passage.

15. The apparatus of claim 14, wherein the clamping device comprises a wing member that can be urged into clamping contact with the elongate member.

16. The apparatus of claim 14, wherein the clamping device comprises a threaded member configured to increase the friction force between the elongate member and the connecting member.

17. A spacer device for use with a primary spinal fixation device, comprising:
a compressible spacer sized to fit between the spinous processes of two vertebrae and configured to reduce the range of motion of at least one vertebra;
a transverse member configured to extend from one side of the midline of the spine, through the interspinous process space, the transverse member being coupled with the spacer; and
a connecting member attachable to the transverse member and to a primary spinal fixation device.

18. The spacer device of claim 17, wherein the compressible spacer is sized to fit between the spinous processes of two lumbar vertebrae.

19. A method for reducing or delaying degenerative disc disease, comprising:
accessing a region of the spine where normal range of motion is compromised;
placing a spacer between a vertebral portion of one of the vertebrae for which the normal range of motion is compromised and a corresponding vertebral portion of an adjacent vertebrae, wherein the spacer is coupled with a fixation assembly by a rod.

20. The method of claim 19, wherein the vertebral portion is a spinous process.

21. The method of claim 19, wherein the vertebral portion is a lamina.

22. The method of claim 19, wherein the spacer is movably coupled using a moveable device to permit some motion of the spacer relative to the vertebrae for which the normal range of motion is compromised.

23. The method of claim 22, wherein the moveable device is a ball joint.

24. The method of claim 19, wherein the spacer is movably coupled using a moveable device to permit some motion of the spacer relative to a motion limiting device coupled with the vertebrae for which the normal range of motion is compromised.

25. The method of claim 24, wherein the moveable device is a ball joint.

26. The method of claim 19, further comprising inserting an access device through a minimally invasive incision in the skin of the patient.

27. The method of claim 26, further comprising expanding said access device from a first configuration to a second configuration, the second configuration having an enlarged cross-sectional area at a distal portion thereof such that the distal portion extends across at least two adjacent vertebrae.
